(19) United States
(12) Patent Application Publication
(10) Pub. No.: US 2020/0015913 A1
(43) Pub. Date: Jan. 16, 2020

(54) FLEXIBLE CIRCUIT BEARING A COIL HAVING A NON-FERROUS CORE

(71) Applicant: Lucent Medical Systems, Inc., Kirkland, WA (US)

(72) Inventor: Curtis S. KING, Kirkland, WA (US)

(21) Appl. No.: 16/509,104

(22) Filed: Jul. 11, 2019

Related U.S. Application Data
(60) Provisional application No. 62/698,766, filed on Jul. 16, 2018.

(51) Int. Cl. A61B 34/20; H01F 27/28; H05K 1/02; H01F 27/32; H01F 41/06

(52) U.S. Cl. CPC A61B 34/20; H01F 27/28; H05K 1/0277; A61B 17/00234; H01F 41/06; A61B 2034/2051; H01F 27/32

(57) ABSTRACT

An electromagnetic coil device includes a non-ferrous core having a substantially cylindrical shape and an insulated conductive medium such as piano wire arranged as a plurality of windings coiled around the non-ferrous core. The insulated conductive medium includes a conductive medium encased within an insulating medium. The plurality of windings include a first portion of windings and a second portion of windings separated by a third portion of windings. The insulating medium has at least one breakdown characteristic that permits shorting between individual conductors of the conductive medium in the first and second portions of windings thereby creating a first conductor region and a second conductor region electrically separated by the third portion of windings.

### BACKGROUND

### Technical Field

The present disclosure generally relates to a flexible circuit device bearing a coil, the coil having a non-ferrous core. More particularly, but not exclusively, the present disclosure relates to a coil structure having, for example, an air core, which is arranged at one end of a flexible circuit, which can be used to track a medical instrument advanced within a body of a patient.

### Description of the Related Art

In many medical procedures, a medical practitioner accesses an internal cavity of a patient using a medical instrument. In some cases, the medical practitioner accesses the internal cavity for diagnostic purposes. In other cases, the practitioner accesses the cavity to provide treatment. In still other cases, different therapy is provided.

Due to the sensitivity of internal tissues of a patient's body, incorrectly positioning the medical instrument within the body can cause great harm. Accordingly, it is beneficial to be able to precisely track the position of the medical instrument within the patient's body. However, accurately tracking the position of the medical instrument within the body can be quite difficult. The difficulties are amplified when the medical instrument is placed deep within the body of a large patient.

One example of technology to track a medical device in the body of a patient is U.S. patent application Ser. No. 15/911,006 to King, which is entitled FLEXIBLE CIRCUIT BEARING A TRACKABLE LOW-FREQUENCY ELECTROMAGNETIC COIL, and which is incorporated herein by reference to the fullest extent allowed by law.

All of the subject matter discussed in the Background section is not necessarily prior art and should not be assumed to be prior art merely as a result of its discussion in the Background section. Along these lines, any recognition of problems in the prior art discussed in the Background section or associated with such subject matter should not be treated as prior art unless expressly stated to be prior art. Instead, the discussion of any subject matter in the Background section should be treated as part of the inventor's approach to the particular problem, which, in and of itself, may also be inventive.

### BRIEF SUMMARY

The use of known technology to track a medical device within the body of the patient is improved when the tracking device is a flexible circuit device bearing a coil that has a non-ferrous core. The device may be easier to manufacture, less expensive to manufacture, smaller, lighter, less visible to certain imaging technologies, and also imbued with various other desirable features.

In a first embodiment, an electromagnetic coil device includes a non-ferrous core having a substantially cylindrical shape and an insulated conductive medium arranged as a plurality of windings coiled around the non-ferrous core. The insulated conductive medium includes a conductive medium encased within an insulating medium. The plurality of windings includes a first portion of windings and a second portion of windings separated by a third portion of windings. The insulating medium has at least one breakdown characteristic that permits shorting between individual conductors of the conductive medium in the first and second portions of windings thereby creating a first conductor region and a second conductor region electrically separated by the third portion of windings.

In at least some cases of the first embodiment, the electromagnetic coil device includes a flexible printed circuit having a length and a width. In these cases, the length is at least twenty times the width. The flexible printed circuit includes a first conductive trace running substantially along the length of the flexible printed circuit and a second conductive trace running substantially along the length of the flexible printed circuit. The first conductive trace has a first end electrically coupled to the first conductor region, and the second conductive trace has a first end electrically coupled to the second conductor region.

In at least some cases of the first embodiment, the breakdown characteristic is a melting point that is below a temperature of liquid solder, and the electrical coupling of the first and second portions to the first and second conductor regions, respectively, is via a solder connection. In these or in some other cases, the breakdown characteristic is a chemical composition that permits separation of the insulating medium from the conductive medium via a chemical reaction, or the breakdown characteristic is a tensile composition that permits separation of the insulating medium from the conductive medium via ultrasound.

In some cases of the first embodiment, the third portion of windings is a multi-layer portion of windings. In these or some other cases, the third portion of windings has a pitch of about 30 to 60 degrees off of an axis of the non-ferrous core. In some cases, the first, second, and third portions of windings form a continuous set of windings, and in some cases, the first portion of windings are electrically coupled to a first end of the third portion of windings via a first conductive conduit, and the second portion of windings are electrically coupled to a second end of the third portion of windings via a second conductive conduit.

In some cases, the third portion of windings has a linear length of between about 0.006 inches and 0.125 inches. In some cases, the third portion of windings has an outside diameter of between about 0.0025 inches and two (2) inches in linear length. In some cases the non-ferrous core is a hollow core. And in some cases, the non-ferrous core is a ceramic core, a resin core, or a glass core.

In a second embodiment, a method of operating a medical device includes passing a distal end of the medical device into a body of a patient while a proximal end of the medical device remains outside the body of the patient. The distal end of the medical device includes a non-ferrous core having a substantially cylindrical shape and an insulated conductive medium arranged as a plurality of windings coiled around the non-ferrous core. The insulated conductive medium includes a conductive medium encased within an insulating medium. The plurality of windings include a first portion of windings and a second portion of windings separated by a third portion of windings. And the insulating medium has at least one breakdown characteristic that permits shorting between individual conductors of the conductive medium in the first and second portions of windings thereby creating a first conductor region and a second conductor region electrically separated by the third portion of windings. The method also includes generating an excitation signal from a current induced in the third portion of windings arranged at the distal end of the medical device by an electromagnetic field, and operating ancillary circuitry arranged at the proximal end of the medical device to detect the excitation signal generated in the third portion of windings. The excitation signal is passed via first and second conductive traces running substantially along a length of a flexible printed circuit. The first conductor region is electrically coupled to a first end of the first conductive trace and the second conductor region is electrically coupled to a first end of the second conductive trace.

In some cases of the second embodiment, based at least in part on the detected excitation signal, the method also includes generating a representation of the distal end of the medical device in the body of the patient and communicating the representation of the distal end of the medical device in the body of the patient to a presentation system. In some of these cases, the method includes advancing the distal end of the medical device further into the body of the patient and tracking the distal end of the medical device as it advances into the body of the patient. What's more, in some cases, passing the distal end of the medical device into the body of the patient includes passing the passing the distal end of the medical device through a lumen of a catheter.

In a third embodiment, a method of manufacturing a medical device includes providing a non-ferrous core having a substantially cylindrical shape and winding an insulated conductive medium around the non-ferrous core a plurality of times. The insulated conductive medium includes a conductive medium encased within an insulating medium. The winding creates a first portion of windings and a second portion of windings separated by a third portion of windings. The method further includes creating a first breakdown condition to exceed a breakdown characteristic of the insulating medium that shorts together individual conductors of the conductive medium in the first portion of windings thereby creating a first conductor region. And the method includes creating a second breakdown condition to exceed the breakdown characteristic of the insulating medium that shorts together individual conductors of the conductive medium in the second portion of windings thereby creating a second conductor region.

In some cases, the method of the third embodiment includes providing a flexible printed circuit having a length and a width, wherein the length is at least twenty times the width. The flexible printed circuit includes first and second conductive traces running substantially along the length of the flexible printed circuit. The method also includes electrically coupling a first end of the first conductive trace to the first conductor region and electrically coupling a first end of the second conductive trace to the second conductor region.

In these or other cases of the third embodiment, creating the first breakdown condition includes soldering the first end of the first conductive trace to the first conductor region and creating the second breakdown condition includes soldering the first end of the second conductive trace to the second conductor region. In some cases, winding the insulated conductive medium around the non-ferrous core the plurality of times includes winding the insulated conductive medium in a plurality of layers.

This Brief Summary has been provided to introduce certain concepts in a simplified form that are further described in detail below in the Detailed

Description. Except where otherwise expressly stated, the Brief Summary does not identify key or essential features of the claimed subject matter, nor is it intended to limit the scope of the claimed subject matter.

### BRIEF DESCRIPTION OF THE SEVERAL VIEWS OF THE DRAWINGS

Non-limiting and non-exhaustive embodiments are described with reference to the following drawings, wherein like labels refer to like parts throughout the various views unless otherwise specified. The sizes and relative positions of elements in the drawings are not necessarily drawn to scale. For example, the shapes of various elements are selected, enlarged, and positioned to improve drawing legibility. The particular shapes of the elements as drawn have been selected for ease of recognition in the drawings. One or more embodiments are described hereinafter with reference to the accompanying drawings in which:

FIG. 1 is a block diagram of a system for detecting the position of a medical instrument within a body of a patient, according to one embodiment;

FIG. 2 is an embodiment of a flexible circuit device bearing an electromagnetic coil structure having a non-ferrous core;

FIG. 3A is a coil embodiment wound over a polyimide tube;

FIG. 3B is a cross-sectional view of the coil embodiment of FIG. 3A taken along line A-A;

FIG. 3C is a magnified portion of FIG. 3B in the area identified as Detail 3C;

FIG. 4 is an electromagnetic coil structure before it is electromechanically coupled to a flexible circuit;

FIG. 5A is the electromagnetic coil structure before it is electromechanically coupled to a flexible circuit;

FIG. 5B is the electromagnetic coil structure in the process of being electromechanically coupled to a flexible circuit;

FIG. 6A is a top view of the flexible circuit device bearing a coil having a non-ferrous core;

FIG. 6B is a side view of the device of FIG. 6A;

FIG. 7A is a cutaway view embodiment of a flexible circuit device bearing an electromagnetic coil structure within a lumen of a catheter during a medical procedure; FIG. 7B is a front-end view of the embodiment of FIG. 7A;

FIG. 8A is an electromagnetic coil structure before it is electromechanically coupled to a flexible circuit;

FIG. 8B is a flexible circuit device embodiment bearing a coil having a non-ferrous core;

FIG. 9A is another electromagnetic coil structure before it is electromechanically coupled to a flexible circuit;

FIG. 9B is a magnified portion of FIG. 9A in the area identified as Detail 9B;

FIG. 10A is another electromagnetic coil structure before it is electromechanically coupled to a flexible circuit;

FIG. 10B identifies a cutaway portion of the electromagnetic coil structure of FIG. 10A;

FIG. 10C magnifies the cutaway portion of FIG. 10B in the area identified as Detail 10C;

FIG. 11A is yet one more electromagnetic coil structure before it is electromechanically coupled to a flexible circuit; and

FIG. 11B is a flexible circuit device embodiment bearing a coil having a non-ferrous core;

FIGS. 12A-12H illustrate formation and use of a flexible circuit device embodiment;

FIGS. 13A-13C illustrate formation and use of yet one more flexible circuit device embodiment.

### DETAILED DESCRIPTION

In the following description, certain specific details are set forth in order to provide a thorough understanding of various disclosed embodiments. However, one skilled in the relevant art will recognize that embodiments may be practiced without one or more of these specific details, or with other methods, components, materials, etc. Also in these instances, well-known structures may be omitted or shown and described in reduced detail to avoid unnecessarily obscuring descriptions of the embodiments.

The present invention may be understood more readily by reference to this detailed description of the invention. The terminology used herein is for the purpose of describing specific embodiments only and is not limiting to the claims unless a court or accepted body of competent jurisdiction determines that such terminology is limiting. Unless specifically defined herein, the terminology used herein is to be given its traditional meaning as known in the relevant art.

In many medical situations, it is desirable to penetrate the solid or semi-solid biological matter of a patient's body, and guide a medical instrument to a precise location. For example, one common medical practice involves diagnosis and therapy of a tumor in a patient's body. Another common medical practice involves accurately placing a flexible catheter in a patient's body. When a portion of the medical instrument (e.g., the flexible catheter) that will penetrate and pass into the patient's body has at least one electromagnet coil structure, and when an excitation signal is induced within the electromagnet coil structure, then the electromagnet coil structure will be trackable to a precise location within the body of the patient.

An electromagnetic field (EMF) generator is operated by a medical practitioner proximal to the body of the patient. In some cases, the medical practitioner places the EMF generator directly in contact with the body of the patient. In some cases, the medical practitioner will attempt to place the EMF generator adjacent to the portion of the patient's body where the electromagnet coil structure is believed to be. In still other cases, the EMF generator is placed in another location in proximity to the patient.

Induction coils in the EMF generator produce electromagnetic fields having known characteristics. The electromagnetic fields induce corresponding excitation signals in the electromagnet coil structure integrated with the medical instrument. The induced excitation signals have measurable current and voltage parameters, which are inversely proportional to the cube of the distance from electromagnetic source. By this relationship, the excitation signals may be measured with ancillary detection circuitry coupled to the electromagnetic coil structure. The changing values representative of the excitation signal are then computationally processed to determine the electromagnet coil structure's location and movement in three dimensional space to an acceptably accurate precision (e.g., within 0.5 millimeters (mm), within 1 mm, within 2 mm, or within some other acceptable distance based on the medical procedure being performed).

The EMF generator may include any number of electromagnetic transmitters to create variable magnetic fields. The variable magnetic fields are created in any desirable way from source signals of various voltages, currents, frequencies, phases, encodings, or other characteristics. As the electromagnet coil structure moves through the variable magnetic fields, the induced excitation signal will change in a detectably corresponding manner.

In some cases, the EMF generator includes three electromagnetic transmitters placed in an equilateral triangle formation, and the EMF generator is substantially located along a midsagittal plane of the area of interest. Such a formation permits the electromagnet coil structure to produce excitation signals suitable for determining the location and movement of the medical instrument in two dimensions.

In other cases, the EMF generator includes at least two sets of three electromagnetic transmitters (i.e., at least six transmitters total) arranged equidistant from each other, and with right angles between them. In this configuration, two electromagnetic coil structures are employed, and at any one time, both of the electromagnet coil structures are never perpendicular to more than three of the electromagnetic transmitters. From this configuration, the excitation signals generated by the electromagnetic coil structure can be used to computationally triangulate the location and orientation of the medical instrument in up to six degrees of freedom (i.e., three dimensional Cartesian coordinates and three axes of rotation).

The electromagnet coil structure can be represented as a dipole with five degrees of freedom (i.e., three dimensional Cartesian coordinates and two angles, which represent the angular alignment of the dipole relative to the electromagnetic transmitter that induced the excitation signal). When the dipole is perpendicular to an electromagnetic transmitter, no excitation signal is generated because the varying magnetic field cancels itself out. Orthogonal placement of three (3) electromagnetic field generators allows for calculation of the position of a single coil electromagnetic coil structure with five (5) degrees of freedom (DOF). A second, non-symmetric, electromagnetic coil structure in this field arrangement allows for calculation of the position and orientation with six (6) DOF. The parameters of the excitation signal measured in detection circuitry that is electrically coupled to the electromagnet coil structures changes as a function of the varying distance and angle between electromagnet coil structures and the axis of each transmitter. The measured parameters are then used to calculate the distance between each electromagnetic transmitter and the electromagnet coil structures. Values (e.g., changes in voltage, current, or voltage and current) for six equations are provided by the detection circuitry, and these are used to calculate the Cartesian coordinates and the bi-angular position of the electromagnet coil structure.

A presentation system includes one or more of a video display, an audio input/output system, a tactile feedback system, or some other presentation mechanism. The presentation system may further include one or more user input interfaces for keyboards, mice, touch screens, buttons, dials, and other like controls. The presentation system may be arranged to provide input information, to receive output information, or to both provide and receive information to or from, as the case may be, any one or more of an electromagnetic field (EMF) generator, control circuitry, detection circuitry, and the like. In some cases, the presentation system 8 is embodied as an ultrasound device, a fluoroscopy device, a magnetic resonance imaging (MRI) device, a computed tomography (CT) device, a computer-assisted surgery device, an augmented reality device, a mobile device (e.g., smartphone, tablet, or the like), or some other medical device.

Embodiments of the presentation system are used to present information representing the position and orientation of the medical instrument by receiving and processing magnetic field information. Magnetic field information is produced when the excitation signal is detected by the electromagnet coil structure. The electromagnet coil structure is tracked as the medical instrument (e.g., flexible catheter) is advanced through the body of the patient. The medical instrument does not need to follow a straight line or any specific pattern in order to be tracked.

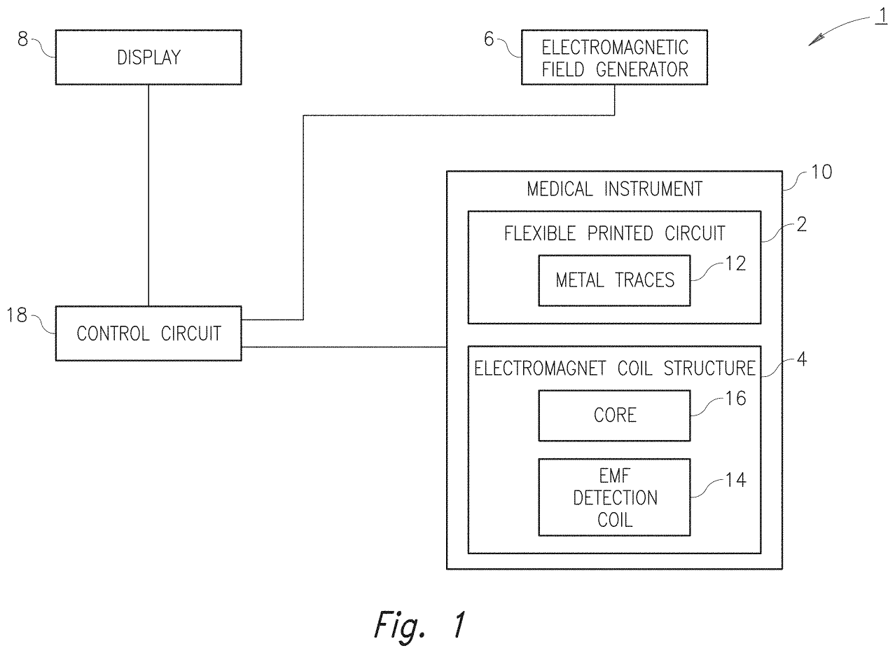

FIG. 1 is a block diagram of a system 1 for detecting the position of a medical instrument 10 within the body of a patient, according to one embodiment. The system 1 includes a medical instrument 10, an electromagnetic field (EMF) generator 6, a presentation system 8, and a control circuit 18. The control circuit 18 is coupled to the medical instrument 10, the EMF generator 6, and the presentation system 8. The medical instrument 10 includes a flexible printed circuit 2, also referred to as a flexible circuit, and an electromagnet coil structure 4. The electromagnet coil structure 4 includes an EMF detection coil 14 wound about a non-ferrous core 16. The flexible printed circuit 2 includes a plurality of metal traces 12 (e.g., two metal traces). The metal traces 12 are electrically coupled to the lead ends of the EMF detection coil 14 so that electrical current induced in the EMF detection coil 14 is passed to detection circuitry of the control circuit 18.

In one embodiment, the medical instrument 10 is a medical device configured to be introduced, either partially or wholly, into the body of a patient in conjunction with a medical procedure. The patient may be a human patient or a non-human patient.

In many cases, the electromagnet coil structure 4 and the flexible printed circuit 2 are integrated with the medical instrument 10. For example, when the medical instrument 10 includes or is a stylet, the electromagnet coil structure 4 and at least a portion of the flexible printed circuit 2 may be formed as part of the stylet.

In many medical procedures, it can be very advantageous to accurately track the position of the medical instrument 10 within the body of the patient. For example, if the medical instrument 10 is delivering fluid to a particular part of the patient's body, then it can be very advantageous to accurately track the position of medical instrument 10 to provide confidence to a medical practitioner that the medical instrument is in the correct position for fluid delivery. In some particularly sensitive medical procedures, knowing the exact position of the medical instrument 10 with substantial certainty can improve the well-being of the patient during a medical procedure.

The EMF generator 6 includes any number of electromagnetic field transmitters that generate a respective magnetic field. The generated magnetic field has characteristics that correspond to a controllable drive signal. The controllable drive signal in FIG. 1 is provided by control circuit 18. The controllable drive signal may have any desired voltage, current, frequency, phase, encoding, polarity, or other characteristics.

The electromagnet coil structure 4 enables tracking of the position of the medical instrument 10. When a current is induced in the EMF detection coil 14, a detectable excitation signal is generated. The excitation signal is representative of the magnetic field generated by the EMF generator 6.

The excitation signal generated by the EMF detection coil 14 is passed through the flexible printed circuit 2 via metal traces 12 to the control circuit 18. The control circuit 18 includes detection circuitry arranged to detect parameters of the excitation signal that represent characteristics of the magnetic field such as field strength and direction. The field strength is inversely proportional to the cube of the distance between the electromagnetic transmitter of the EMF generator 6 and the EMF detection coil 14. Using the parameter values provided by the detection circuitry, the control circuit 18 generates position information of the medical instrument 10, which may include any one or more of location information (e.g., two-dimensional position, three-dimensional position, or the like), orientation, motion, and other location-based information. The position information may then be communicated to a presentation system 8, a database, or any other structure.

In one embodiment, the control circuit 18 both drives a signal to the EMF generator 6 and calculates location-based information (e.g., position, orientation, motion, and the like) of the medical instrument 10. The control circuit 18 receives one or more excitation signals from the medical instrument 10 and analyzes the one or more excitation signals. The control circuit 18 generates the location-based information, such as the position of the medical instrument 10, based on the one or more excitation signals.

In some embodiments, the control circuit 18 may be separate from the medical instrument 10. In other embodiments, the control circuit 18 may be integrated into the medical instrument 10.

In some embodiments, the control circuit 18 may be separate from the EMF generator 6. In other embodiments, the control circuit 18 may be integrated into the EMF generator 6.

In one embodiment the control circuit 18 executes particular algorithms to identify and track the position of the medical instrument 10 in three dimensions and the orientation of medical instrument 10 relative to a reference point, based on the position of the electromagnet coil structure 4. In these and other cases, tracking the position of the medical instrument 10 includes integrating current and historical position data in order to predict one or more future positions of the medical instrument 10.

It can be difficult to accurately track the position of the medical instrument 10 within the body of the patient as the medical instrument 10 is positioned deeper within the body of the patient. In larger patients, the problem can be exacerbated because the medical instrument 10 may need to travel deeper below the skin of the patient in order to reach particular areas of the body in accordance with various medical procedures. It can be difficult to generate a magnetic field with sufficient strength and stability to allow reliable tracking of the medical instrument 10. This problem can be compounded by the fact that in many circumstances it is more desirable to have an EMF detection coil 14 and a core 16 that are relatively small, in order to reduce disruption of body tissues as the medical instrument 10 is introduced into the body of the patient. As the dimensions of the conductive coil 14 are reduced, it can be difficult to generate sufficiently strong and acceptably stable excitation signals. Furthermore, interference from the Earth's magnetic field, from other medical and non-medical equipment that may be positioned in or near the patient's body, and from the medical instrument 10 itself can make it difficult to accurately calculate the position of the medical instrument 10 within the body of the patient.

In one embodiment, in order to enable more accurate tracking of the medical instrument 10 deep within the body of a patient, the control circuit 18 drives the EMF generator with a low frequency drive signal instead of a DC signal or a high-frequency drive signal. The low-frequency drive signal causes a current to be passed through an electromagnetic transmitter. As the direction and magnitude of the current change, the parameters of the magnetic field generated by the electromagnetic transmitter also change. The magnetic field generated by the electromagnetic transmitter has particular characteristics based in part on the waveform of the drive signal. These particular oscillating characteristics can enable the detection circuitry coupled to the EMF detection coil 14 to distinguish the magnetic field generated by the EMF generator 6 from noise, interference, and/or other magnetic fields. In this way, the control circuit 18 can track the position of the medical instrument 10 with acceptable accuracy, even when the medical instrument 10 is deep within the body of the patient.

The control circuit 18 may drive the EMF generator 6 with a drive signal of any frequency or a plurality of frequencies. In one embodiment, the control circuit 18 drives the EMF generator 6 with a drive signal having a frequency of 1000 Hz to 100,000 Hz. The excitation may be selected specifically to avoid AC line related components, which might occur at a multiple of a line frequency. For example, 3000 Hz, which is a multiple of both 50 Hz and 60 Hz—two common line frequencies in Europe and the U.S., respectively—may provide strong magnetic returns, but the strong magnetic returns may also have measurable harmonic components associated with the AC line frequency.

The control circuit 18 has been described as driving the EMF generator 6 with a drive signal. The control circuit 18 can accomplish this by directly applying the drive signal to a conductive coil of the EMF generator 6. Alternatively, the control circuit 18 can accomplish this indirectly by controlling a voltage source to apply a voltage to the EMF generator 6 or by controlling a current source to supply a current to the EMF generator 6. Those of skill in the art will recognize, in light of the present disclosure, that the control circuit 18 can apply a drive signal to the EMF generator 6 in many other ways. All such other ways fall within the scope of the present disclosure.

In one embodiment, the presentation system 8 displays a visual representation of the position of the medical instrument 10 within the body of the patient. The visual representation of the position of the medical instrument 10 enables medical personnel to accurately know the position of the medical instrument 10 within the body of the patient. This in turn can enable the medical personnel to correctly perform medical procedures on the patient.

In one embodiment, the control circuit 18 generates a video signal, and outputs the video signal to the presentation system 8. The video signal includes a representation of the position of the medical instrument 10 within the body of the patient. The video signal can also include position data that can be displayed on the presentation system 8. The position data can include text that indicates numerical coordinates representing the position, orientation, and motion of the medical instrument 10. The presentation system 8 can display both the visual representation of the position of the medical instrument 10 within the body of the patient and the position data indicating the position of the medical instrument 10 within the body of the patient.

The control circuit 18 may include multiple discrete control circuit portions. The control circuit 18 can include one or more microcontrollers, one or more microprocessors, one or more memory devices, one or more voltage sources, one or more current sources, one or more analog-to-digital converters, one or more digital-to-analog converters, and/or one or more wireless transceivers. One or more of these components can collectively make up the control circuit 18.

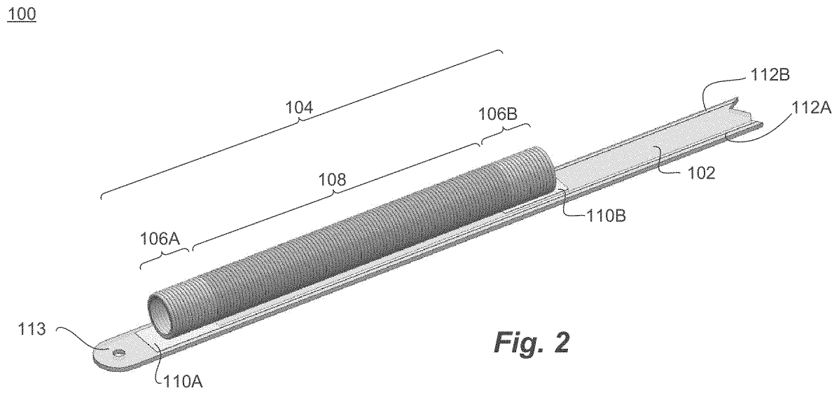

FIG. 2 is an embodiment of a flexible circuit device 100 bearing a substantially cylindrical electromagnetic coil structure 104 having a non-ferrous core. A flexible circuit substrate 102 of desirable length, width, and material is also provided. In at least some embodiments, the length of the flexible circuit is at least twenty times the width. The electromagnetic coil structure 104 is arranged at a distal end of the flexible circuit substrate 102. In some cases, the electromagnetic coil structure 104 is positioned at about the end (e.g., within 25 millimeters (mm) of the end) of the flexible circuit substrate 102. In some cases, the electromagnetic coil structure 104 is positioned at about the middle of the flexible circuit substrate 102. In other cases, the electromagnetic coil structure 104 is positioned in some other location on the flexible circuit substrate 102. In still other cases, multiple electromagnetic coil structures 104 are positioned at multiple locations on the flexible circuit substrate 102.

The electromagnetic coil structure 104 of FIG. 2 is a substantially cylindrical, continuously formed, conductive coil. The electromagnetic coil structure 104 includes three distinct portions: a first portion 106A and a second portion 106B are separated by a third portion 108. The first portion 106A is electromechanically coupled to a first conductor region 110A, and the second portion 106B is electromechanically coupled to a second conductor region 110B. First and second ends of the third portion 108 are electrically coupled to the first portion 106A and the second portion 106B, respectively, but the central body of the third portion 108 is electrically insulated from the flexible circuit substrate 102 and the traces integrated therein. In this way, the electromagnetic coil structure 104 is formed as an electrically conductive coil having a distal area of electrical coupling via the first portion 106A and a proximal point of electrical coupling via the second portion 106B. Electrical signals (e.g., electric current, electric voltage, or the like) are passed from the first and second conductor regions 110A, 110B via first and second conductive traces 112A, 112B.

The flexible substrate 102 may comprise a flexible structure (e.g., polymer film, polyimide film, polyester film, plastic film, or the like) laminated or otherwise affixed to a conductive material such as a thin sheet of copper. The conductive material may be etched or otherwise formed to produce first and second conductor regions 110A, 110B, first and second conductive traces 112A, 112B, and any other circuit patterns if so desired. The conductive patterns may be formed on one or more sides of the flexible structure (e.g., top-side, bottom-side, inside), and in some cases, the conductive patterns may be layered between insulating flexible structures, shielding structures, and other structures. An overcoat (e.g., insulating, protective polymer) may be formed on the flexible substrate 102 before components are added, after components are added, or before and after components are added.

In some embodiments, the flexible substrate 102 may be polyimide film, though other materials are also contemplated. The flexible substrate 102 may be selected based on any one or more particular properties. A non-limiting, non-exhaustive list of properties includes a resistance to high heat (e.g., greater than 400 degrees Fahrenheit), electrical resistance, dimensional stability, dielectric strength, flexural capability, and durability.

In FIG. 2, an aperture (e.g., hole) passing through the flexible substrate 102 in the distal section is illustrated. The aperture 113 may be located in any other selected location. In other embodiments, a metal feature (e.g., solder pad) may be formed or otherwise arranged in the distal section or some other section of the flexible substrate 102. In still other embodiments, a one or more different features may be formed, integrated, attached, or the like. The hole, metal pad, or other features may be used for assembly of the flex substrate 102 within a medical device or for any other desired purpose. For example, in some cases, the hole is arranged to mate with a pin, tab, or some other attachment feature integrated with or otherwise associated with the medical device. Other methods/features are also contemplated.

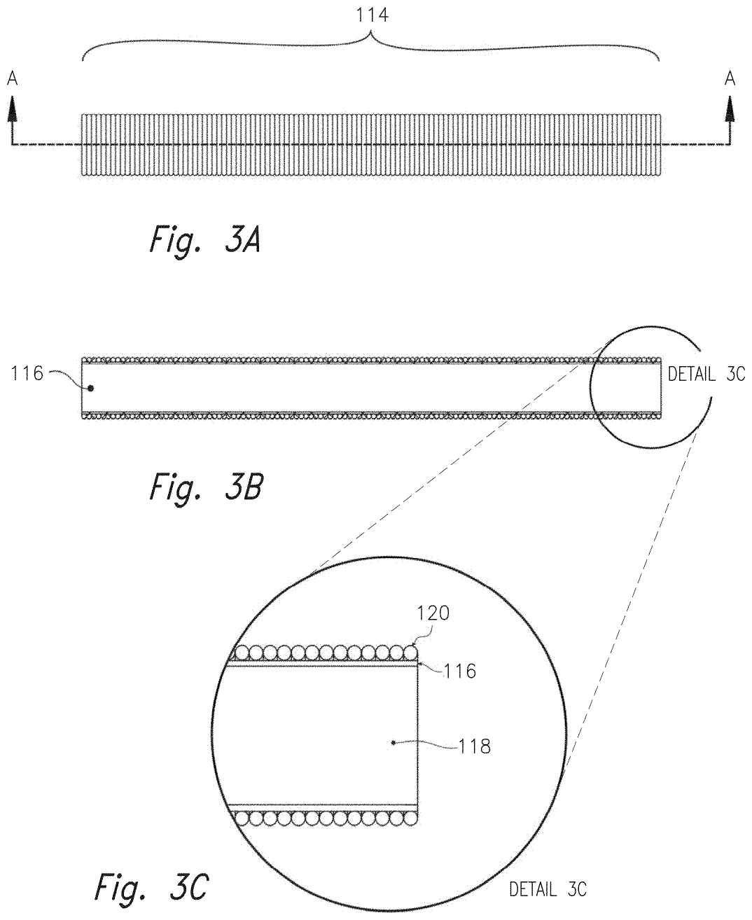

FIG. 3A is a coil 114 embodiment wound over a non-ferrous core 116. FIG. 3B is a cross-sectional view of the coil 114 of FIG. 3A taken along line A-A. And FIG. 3C is a magnified portion of FIG. 2B in the area identified as Detail 3C. In the present disclosure, FIGS. 3A-3C may be collectively referred to as FIG. 3.

The length of coil 114 may be about 25 mils (i.e., 0.0250 inches) to about 4000 mils (i.e., four inches). In other embodiments, coils formed along the lines of coil 114 may be shorter than 25 mils or longer than 4000 mils. In at least one case, coil 114 has a length of about 50 mils. In at least one other case, coil 114 has a length of about two inches.

The outside diameter of coil 114 may be about three mils (i.e., 0.0030 inches) to about 500 mils (i.e., 0.5000 inches). In other embodiments, coils formed along the lines of coil 114 may have smaller outside diameters than three mils or larger outside diameters than 500 mils. In at least one case, coil 114 has an outside diameter of about 6 mils. In at least one other case, coil 114 has an outside diameter of about 250 mils.

The coil 114 of FIG. 3 is wound around a polyimide tube (i.e., non-ferrous core 116). The non-ferrous core 116 is about the same length as the coil 114. In some embodiments, however, the non-ferrous core 116 may be longer than the coil 114 by any desired amount.

The outside diameter of non-ferrous core 116 may be about 2.5 mils (i.e., 0.0025 inches) to about 400 mils (i.e., 0.4000 inches). In other embodiments, non-ferrous cores formed along the lines of coil 114 may have smaller outside diameters than 2.5 mils or larger outside diameters than 400 mils. In at least one case, non-ferrous core 116 has an outside diameter of about 5 mils. In at least one other case, non-ferrous core 116 has an outside diameter of about 200 mils.

Coil 114 is realized by winding a conductive medium 120 around a physical core. They physical core may be substantially cylindrical. The physical core may have a circular cross-section, square cross-section, hexagonal cross-section, octagonal cross-section, or a cross-section having any other desirable shape. Even in cases where the physical core has a non-circular cross-section, the physical core may be referred to as substantially cylindrical.

The conductive medium 120 is a ferrous material such as piano wire (e.g., steel wire, copper wire, or the like) encapsulated by an insulating medium. The diameter of conductive medium 120 may be about one mil (i.e., 0.0010 inches) to about 100 mils (i.e., 0.1000 inches). In some embodiments, conductive medium 120 used in coils formed along the lines of coil 114 may have smaller outside diameters than one mil or a larger outside diameter than 100 mils. In at least one case, conductive medium 120 has an outside diameter of about 2 mils. In at least one other case, conductive medium 120 has an outside diameter of about 50 mils. The conductive medium 120 may have a circular cross-section, square cross-section, hexagonal cross-section, octagonal cross-section, rectangular cross-section, or a cross-section having any other desirable shape.

The insulating medium of conductive medium 120 may be selected based on any one or more of its resistance to high heat (e.g., greater than 400 degrees Fahrenheit), electrical resistance, dimensional stability, dielectric strength, flexural capability, and durability. Other reasons for selecting a particular insulating medium for conductive medium 120 are also contemplated. The insulating medium may be a nylon resin, a polymer such as a fluoropolymer or polyimide, a polyvinyl chloride, polyethylene, cross linked polyethylene, or any suitable material.

Each of the reasons for selecting a particular insulating medium may be considered in respect to an associated breakdown condition. One breakdown condition, for example, may be exceeding a particular temperature. If the insulating material is subjected to a temperature that exceeds the insulating medium's resistance to heat, then a breakdown condition occurs, which will cause the insulating medium to fail (e.g., melt, separate, fragment, burn, or the like). Another exemplary breakdown condition may be exceeding a flexural capability. If the insulating material is wound into a too small diameter, then a breakdown condition occurs, which will cause the insulating medium to fail in a different way (e.g., crack, tear, fragment, or the like). Still another breakdown condition is a chemical composition that permits separation of the insulating medium from the conductive medium via a chemical reaction. And yet one more breakdown condition is a tensile composition that permits separation of the insulating medium from the conductive medium via ultrasound or some other like means.

In at least some cases, both the insulating medium and the non-ferrous core 116 may have a set of corresponding breakdown characteristics that occur under different breakdown conditions. For example, the insulating medium and the non-ferrous core 116 may both be selected at least in part based on a resistance to heat. The insulating medium may have a breakdown condition at a first temperature and the non-ferrous core 116 may have a breakdown condition at a second temperature that is higher than the first temperature. Then, when a first breakdown condition (e.g., a soldering temperature) is arranged, the insulating medium breaks down, but because the second breakdown condition (e.g., a temperature higher than the soldering temperature) is avoided, the integrity of the non-ferrous core is maintained without breaking down.

In FIG. 3, the non-ferrous core 116 has a hollow area 118, which may be referred to as a lumen. In some cases, coil 114 is formed over a physical core. The physical core may be a single or multi-layer tube, a filament, a plurality of filaments, or some other form comprising a ferrous or non-ferrous material (e.g., polyimide, polyimide, polytetrafluoroethylene (PTFE), phenolic, cardboard, polyester, stainless steel, brass, ceramic, glass, thermoset resin, or the like). In some cases the physical core is removed after coil 114 is formed. In these cases, non-ferrous core 116 is realized as an air core after the coil is wound around the physical core and after the physical core material is removed.

Considering embodiments of the structures represented in FIGS. 2 and 3, the coil 114 of FIG. 3 may be further processed to form the electromagnetic coil structure 104 of FIG. 2. For example, the first portion 106A of the electromagnetic coil structure 104 may be formed by dipping a first end of coil 114 into a vessel of molten solder, and the second portion 106B of the electromagnetic coil structure 104 may be formed by dipping the opposing second end of coil 114 into the same or a different vessel of molten solder. The molten solder may have a temperature in the range of about 190 degrees Fahrenheit to about 840 degrees Fahrenheit. In at least one embodiment, the molten solder is about 500 degrees Fahrenheit.

When the end of the coil 114 is dipped (e.g., immersed, sunk, submerged, soaked, or the like) in the solder, the high temperature is sufficient to exceed a breakdown condition of the insulating medium of coil windings that are subject to the dipping process and thereby fuse (e.g., electrically short) those coil windings into a large conductor region (e.g., electrical contact area) for that particular end of the coil 114. When these acts are completed for both ends of coil 114, the electromagnetic coil structure 104 is achieved.

In some embodiments, as an alternative or in addition to dipping both ends of the coil 114 into a vessel of molten solder, the flexible circuit device 100 is formed when coil 114 is placed in physical contact with the flexible circuit substrate 102 having the first and second conductor regions 110A, 110B. In these cases, the first portion 106A is heated to a point that creates a breakdown condition (i.e., melting or otherwise breaking down an insulating medium (e.g., encapsulation layer) of the conducting medium (e.g., wire)) and applying an electrically conductive solder to the first portion 106A. When the heat is removed, the solder will harden and thereby fuse the subject coil windings into a permanent electromechanical bond with the first conductive region 110A. Along these lines, the second portion 106B is also formed. That is, the second end of coil 114 is heated to a point that creates the breakdown condition, an electrically conductive solder is applied, and when the heat is removed, the solder will harden and thereby fuse the second portion of coil windings into a permanent electromechanical bond with the second conductive region 110B.

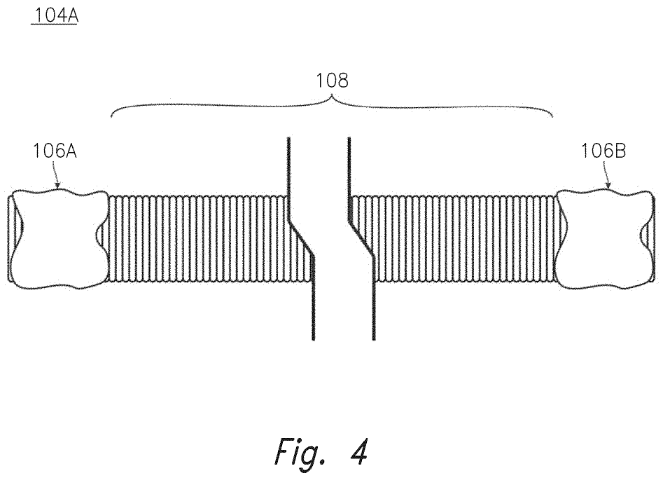

FIG. 4 is an electromagnetic coil structure 104A before the structure is electromechanically coupled to a flexible circuit 102 (FIG. 2). The electromagnetic coil structure 104A includes a first portion 106A, which is a tinned end of a coil, a second portion 106B, which is also a tinned end of the coil, and a third portion 108 between the first and second tinned ends. The third portion 108 can be any desirable length. When placed into operation, the third portion 108 is the active portion of the electromagnet.

The electromagnetic coil structure 104A is formed by providing a non-ferrous core (e.g., a polyimide tube) having a substantially cylindrical shape, and winding an insulated conductive medium (e.g., magnet wire) around the non-ferrous core a plurality of times. The insulated conductive medium includes a conductive medium encased within an insulating medium, and the winding creates a first portion of windings and a second portion of windings separated by a third portion of windings, which portions will become, respectively, the first portion 106A of windings of electromagnetic coil structure 104, the second portion 106B of windings of electromagnetic coil structure 104, and the third portion 108 of windings of electromagnetic coil structure 104. A first breakdown condition (e.g., a solder-tinning temperature) is created to exceed a breakdown characteristic (e.g., melting point) of the insulating medium, which causes individual conductors of the conductive medium in the first portion of windings to short together thereby creating a first conductor region 106B. And a second breakdown condition is created to exceed the breakdown characteristic (e.g., melting point) of the insulating medium that shorts together individual conductors of the conductive medium in the second portion of windings thereby creating a second conductor region 106B

In at least one embodiment of the electromagnetic coil structure 104A of FIG. 4, the third portion of windings has a linear length of between about 0.006 inches and 0.125 inches, the third portion of windings has an outside diameter of between about 0.0025 inches and two (2) inches in linear length, and the non-ferrous core is a hollow core, a ceramic core, a resin core, or a glass core.

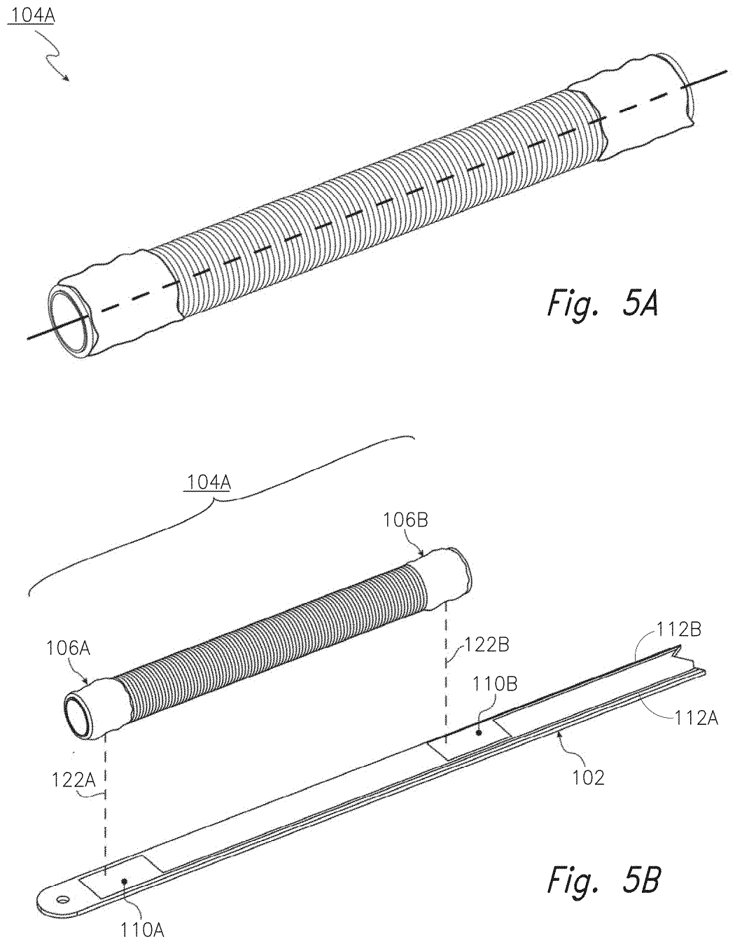

FIG. 5A is the electromagnetic coil structure 104A before it is electromechanically coupled to a flexible circuit 102. A center-line representing a central axis of the electromagnetic coil structure 104A is shown. FIG. 5B is the electromagnetic coil structure 104A in the process of being electromechanically coupled to a flexible circuit 102. FIGS. 5A-5B may be collectively referred to as FIG. 5.

In FIG. 5, the electromagnetic coil structure 104A is provided along alignment tracks 122A, 122B after formation of the electromagnetic coil structure 104. The alignment tracks may be followed manually. Alternatively, the alignment tracks 122A, 122B may be followed in an automated process after forming the electromagnetic coil structure 104. After the electromagnetic coil structure 104A is directly contacting the flexible substrate 102 at first and second conductor regions 110A, 110B, solder is applied at the first and second portions 106A, 106B. The electromechanical solder coupling may be manually performed, performed via a solder re-flow process, performed via a solder bar, or performed in some other way.

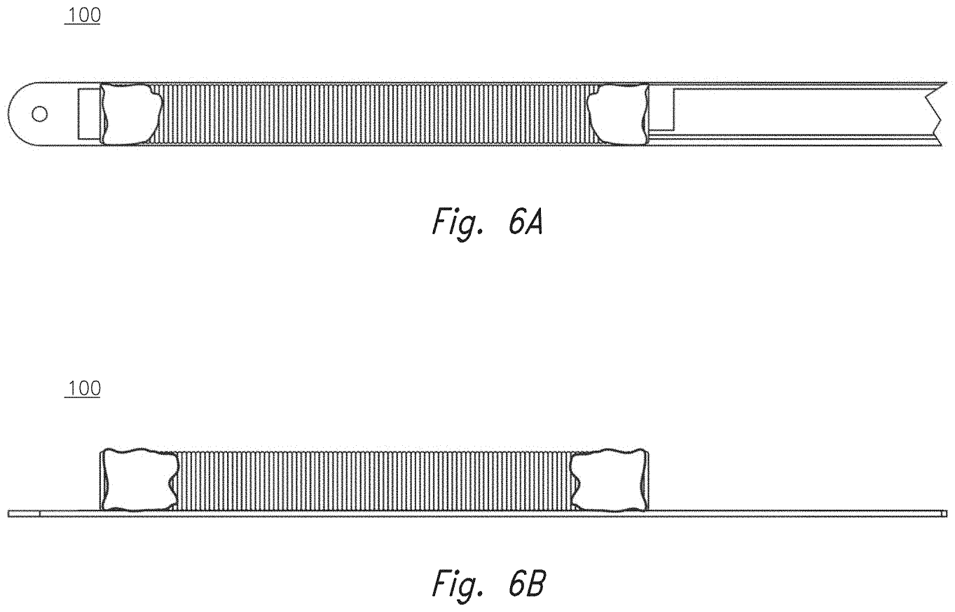

FIG. 6A is a top view of the flexible circuit device 100 bearing a coil having a non-ferrous core, and FIG. 6B is a side view of the device of FIG. 6A. FIGS. 6A-6B may be collectively referred to as FIG. 6.

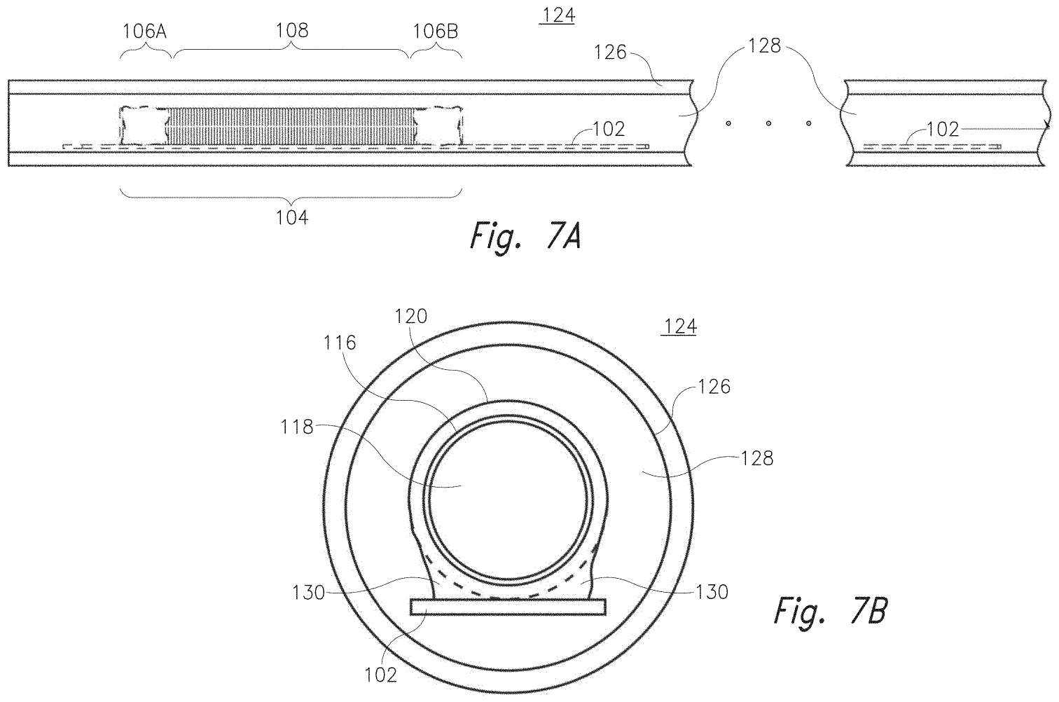

FIG. 7A is a cutaway view 124 embodiment of a flexible circuit device 100 bearing an electromagnetic coil structure 104 within a lumen 128 of a catheter 126 during a medical procedure. FIG. 7B is a front-end view of the embodiment of FIG. 7A. FIGS. 7A-7B may be collectively referred to as FIG. 7.

In FIG. 7, the flexible circuit device 100 includes an electromagnetic coil structure 104 with a single layer of windings of the conductive medium 120. It is recognized that an electromagnetic coil structure embodiment may have a plurality of layers of windings of the conductive medium 120. In the front-end view of FIG. 7B, solder 130 is evident in the solder joint that electromechanically couples the electromagnetic coil structure 104 to the conductive regions of the flexible substrate 102.

FIG. 7 illustrates a method of operating the flexible circuit device 100 in a medical procedure. A medical practitioner may determine that a patient's health will be improved or otherwise served if a form of therapy is directly applied at a location inside the patient's body. The medical practitioner may further determine that the outcome of the medical procedure will be improved if the location where the therapy will be applied is reached with improved precision. For this reason, the medical practitioner chooses to implement the medical procedure with use of a medical device such as the flexible circuit device 100 and a magnetic field sensing device.

To carry out the medical procedure, the medical practitioner passes a distal end of the flexible circuit device 100 into the body of the patient while the proximal end of the flexible circuit device 100 remains outside of the patient's body. As represented in the figures and description herein, the flexible circuit device 100 includes a non-ferrous core having a substantially cylindrical shape and an insulated conductive medium arranged as a plurality of windings coiled around the non-ferrous core, wherein the insulated conductive medium includes a conductive medium encased within an insulating medium. The plurality of windings includes a first portion of windings and a second portion of windings separated by a third portion of windings. The insulating medium has at least one breakdown characteristic (e.g., a particular temperature threshold) that permits shorting between individual conductors of the conductive medium in the first and second portions of windings when the breakdown characteristic is crossed (e.g., exceeding a melting point of the insulating medium). Crossing the breakdown characteristic at particular ends of the coil windings (i.e., in the first and second portions) creates a first conductor region and a second conductor region electrically separated by the third portion of windings.

The non-ferrous core, together with the plurality of coiled windings, forms an electromagnetic coil structure 104 that is electromechanically coupled to a flexible circuit substrate 102. The flexible substrate has a first conductor region 110A electrically coupled to a first end of a first conductive trace 112A and a second conductor region 110B is electrically coupled to a first end of a second conductive trace 112B.

The medical practitioner may advance the flexible circuit device 100 into the patient's body by passing the device inside the lumen 128 of a catheter 126. As the medical practitioner passes at least a portion of the flexible circuit device 100 into the body of the patient, the medical practitioner causes an electromagnetic field (EMF) generator to generate one or more electromagnetic fields in proximity to the patient. Concurrently, ancillary circuitry arranged at the proximal end of the medical device to be operated and thereby detect an excitation signal through the third portion of windings arranged at the distal end of the flexible circuit device 100. The excitation signal is passed via first and second conductive traces running substantially along the length of the flexible printed circuit. Based at least in part on the detected magnetic field and induced excitation signal, a representation of the distal end of the flexible circuit device 100 in the body of the patient is generated and communicated to a presentation system. In this way, as the distal end of the flexible circuit device 100 is advanced further into the body of the patient, the distal end can be tracked.

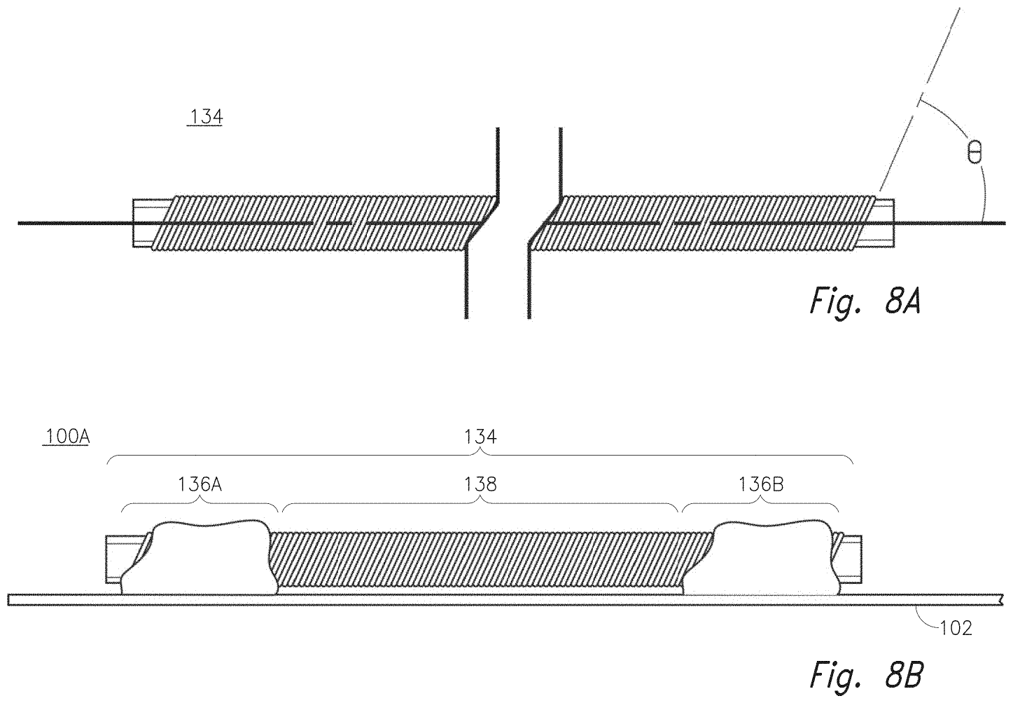

FIG. 8A is an electromagnetic coil structure 134 before it is electromechanically coupled to a flexible circuit 102. FIG. 8B is a flexible circuit device 100A embodiment bearing a coil having a non-ferrous core. The flexible circuit device 100A shows the electromagnetic coil structure 134 of FIG. 8A having now been electromechanically coupled to a flexible circuit 102. FIGS. 8A-8B may be collectively referred to as FIG. 8.

A center-line representing a rotational axis of the electromagnetic coil structure 134 is shown in FIG. 8A. The coil structure in FIG. 8A is canted by an amount equal to angle theta (θ), which may be between zero and ninety degrees (0° to 90°). In at least one embodiment, the coil structure (e.g., a third portion of windings) has a pitch of about 30 to 60 degrees off of an axis of the non-ferrous core. A canted coil such illustrated in FIG. 8 may be selected for many reasons. For example, a canted coil may produce a different electrical response to a generated magnetic field than that of a non-canted coil to the same field. This different electrical response may be detectable and distinguishable by an ancillary device and thereby provide additional information about the electromagnetic coil structure related to its position, orientation, rotation, or any combination thereof. Such information may also be used to distinguish one electromagnetic coil structure from another when a plurality of coils are in concurrent operation. Another reason that a canted coil may be desired is for ease in manufacturing. Still other reasons are also contemplated.

In FIG. 8B, the electromagnetic coil structure 134 is electromechanically affixed to the flexible substrate 102 at first and second conductor regions (not shown), which may be along the lines of first and second conductor regions 110A, 110B in FIG. 5B. Having been so formed, the electromagnetic coil structure 134 may be realized and described as having a first portion of windings 136A and a second portion of windings 136B separated by a third portion of windings 138. The first and second portions of windings 136A, 136B have been soldered to the flexible circuit 102 in first and second conductor regions, respectively. The third portion of windings is operated as the active portion of the electromagnetic coil structure 134.

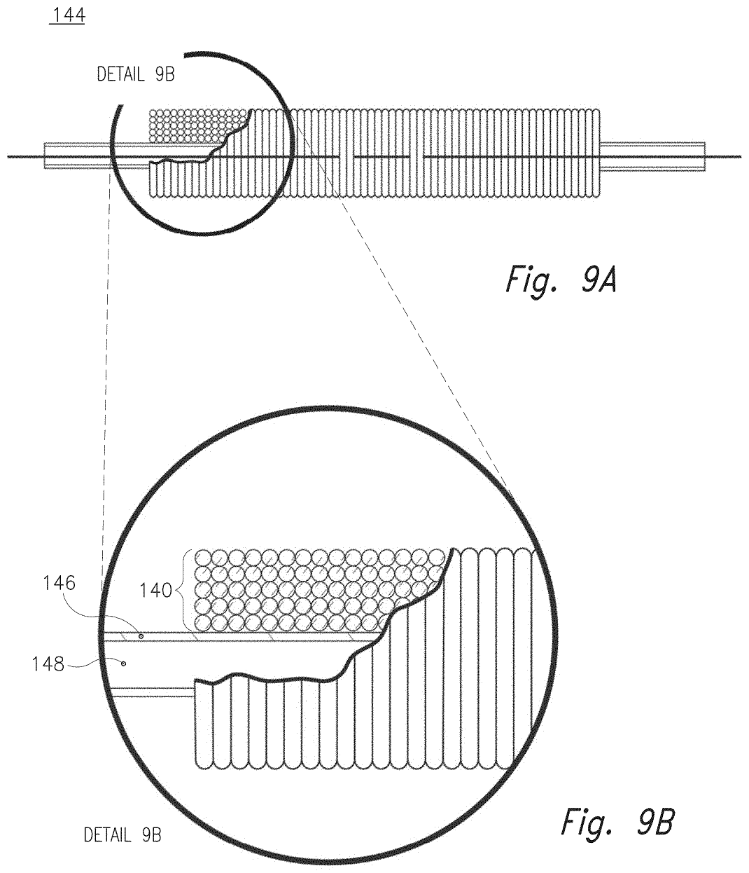

FIG. 9A is another electromagnetic coil structure 144 before it is electromechanically coupled to a flexible circuit. FIG. 9B is a magnified portion of FIG. 9A in the area identified as Detail 9B. FIGS. 9A-9B may be collectively referred to as FIG. 9.

A center-line representing a central axis of the electromagnetic coil structure 144 is shown in FIG. 9A. The coil structure in FIG. 9 has a plurality of layers of coil windings 140 as evident in Detail 9B. The plurality of layers of coil windings 140 are wound on a non-ferrous core 146 of the type described in the present disclosure. A center portion 148 of the non-ferrous core 146 may be a hollow area, an area filled with any desirable medium, or the center portion 148 may comprise the same material as the non-ferrous core 146. In this way, the non-ferrous core 146 may be realized as a hollow tube, a solid filament, or an arrangement of some other suitable composition.

In FIG. 9, the coil windings 140 are shown as being uniformly “stacked” in an array-like arrangement. Other embodiments will nest or otherwise arrange individual coil windings in different orientations relative to each other. In some cases, one arrangement or another may be chosen based on ease of manufacturing, overall outside diameter of the final electromagnetic coil structure, desired electrical performance of the final electromagnetic coil structure, properties of a magnetic field detected by the device, or for other reasons.

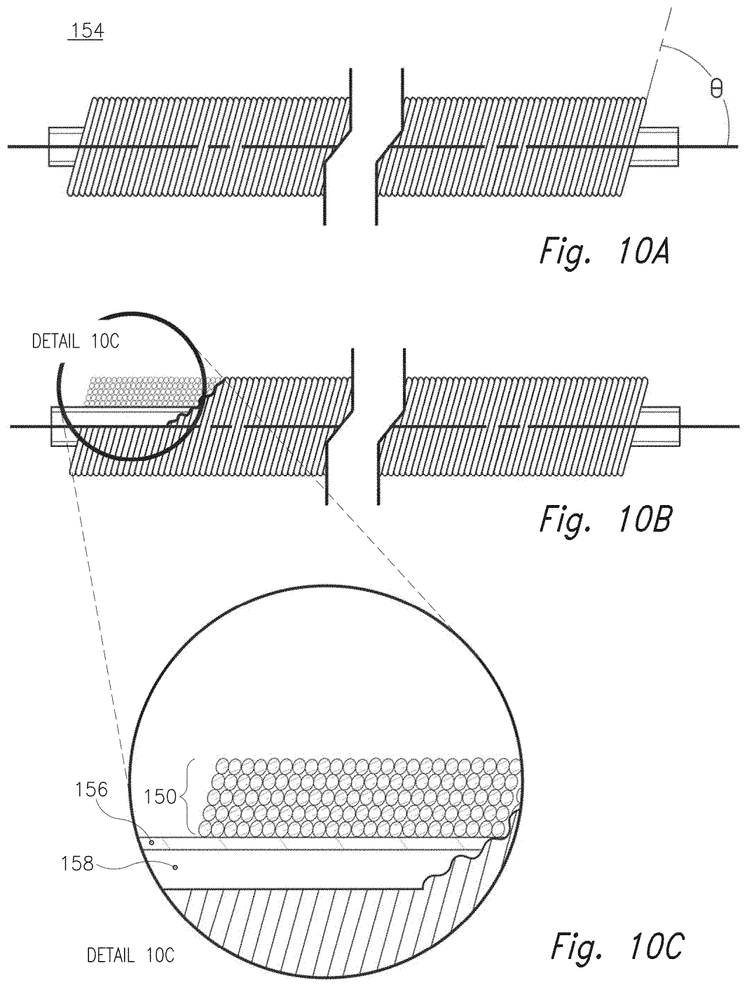

FIG. 10A is another electromagnetic coil structure 154 before it is electromechanically coupled to a flexible circuit. FIG. 10B identifies a cutaway portion of the electromagnetic coil structure 154 of FIG. 10A. And FIG. 10C magnifies the cutaway portion of FIG. 10B in the area identified as Detail 10C. FIGS. 10A-10C may be collectively referred to as FIG. 10.

FIG. 10 is along the lines of electromagnetic coil structure embodiments of FIGS. 8 and 9. A center-line representing a central, rotational axis of the electromagnetic coil structure 154 is shown in FIGS. 10A and 10B. The coil structure in FIG. 10 has a plurality of canted layers of coil windings 150 as evident in Detail 10C. The plurality of canted layers of coil windings 150 are wound on a non-ferrous core 156 of the type described in the present disclosure. A center portion 158 of the non-ferrous core 156 may be a hollow area, an area filled with any desirable medium, or the center portion 158 may comprise the same material as the non-ferrous core 156. In this way, the non-ferrous core 156 may be realized as a hollow tube, a solid filament, or an arrangement of some other suitable composition.

The multi-layer coil structure in FIG. 10 is canted by an amount equal to angle theta (θ), which may be between zero and ninety degrees (0° to 90°). Along the lines of the electromagnetic coil structure 134 of FIG. 8, the canted coil structure illustrated in FIG. 10 may be selected for many reasons (e.g., a desired electrical response to an applied electromagnetic field, an opportunity to capture additional information related to position, orientation, rotation, or any combination thereof, an opportunity to distinguish one medical device from another, ease of manufacture, and at least some other contemplated reasons).

In FIG. 10, the coil windings 150 are shown as being uniformly “stacked” in a canted array-like arrangement. Other embodiments will nest or otherwise arrange individual coil windings in different orientations relative to each other. In some cases, one arrangement or another may be chosen based on ease of manufacturing, overall outside diameter of the final electromagnetic coil structure, properties of a magnetic field produced by the device, or for other reasons.

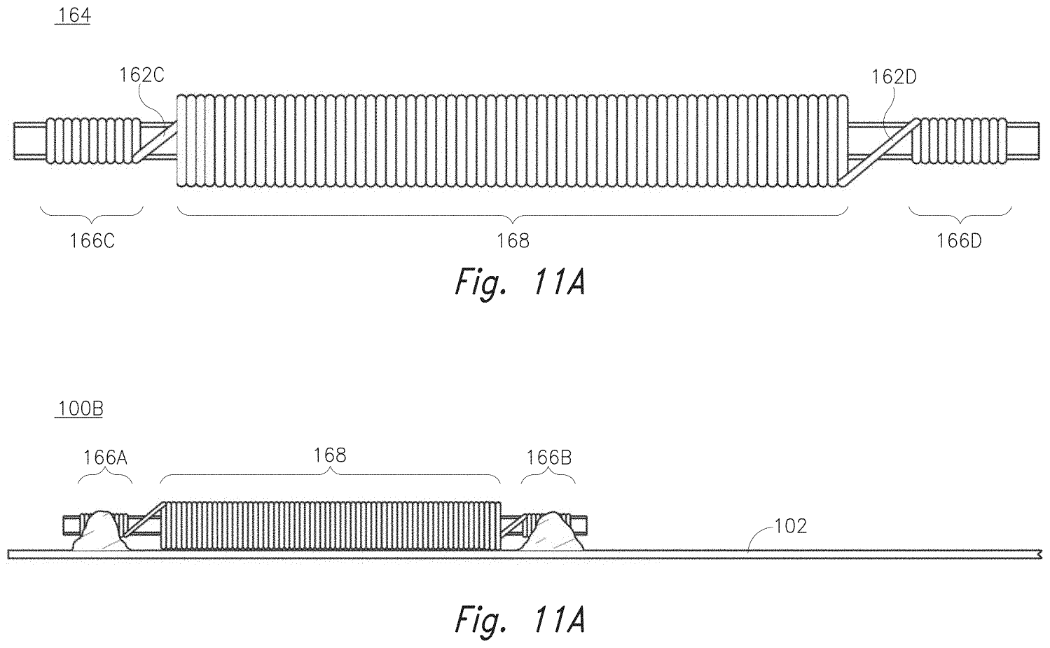

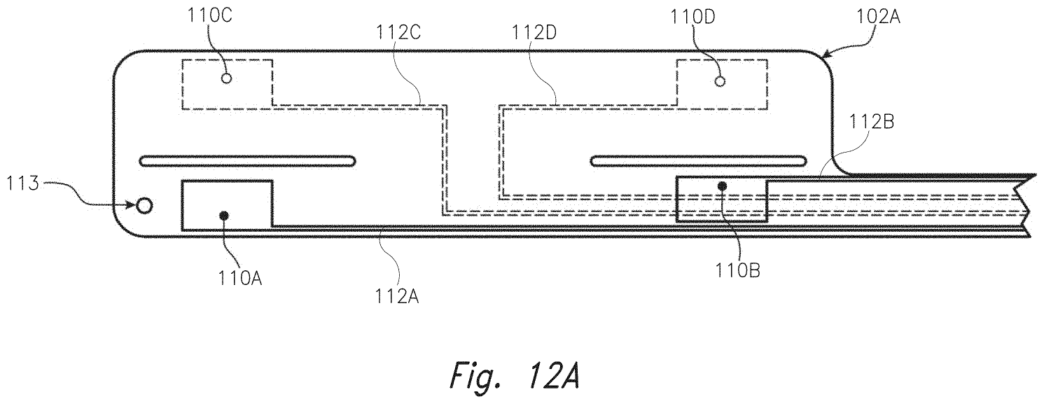

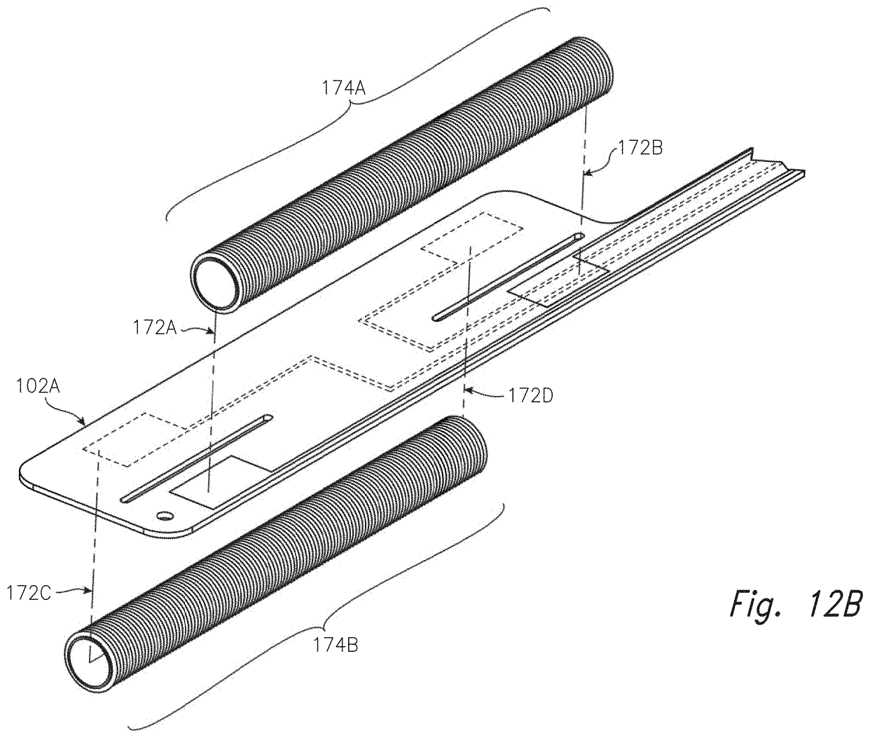

FIG. 11A is yet one more electromagnetic coil structure 164 before it is electromechanically coupled to a flexible circuit 102. FIG. 11B is a flexible circuit device 100B embodiment bearing a coil having a non-ferrous core. The flexible circuit device 100B shows the electromagnetic coil structure 164 of FIG. 11A having now been electromechanically coupled to a flexible circuit 102. FIGS. 11A-11B may be collectively referred to as FIG. 11.

In FIG. 11A, a first portion of windings 166C of electromagnetic coil structure 164 and a second portion of windings 166D of electromagnetic coil structure 164 are separated by a third portion of windings 168. In some embodiments, the third portion of windings 168 are formed from multiple layers of a conductive medium. In some embodiments, the third portion of windings 168 are formed from a single layer of a conductive medium wound on a non-ferrous core having larger diameter. In these or still other embodiments, the third portion of windings are canted. The first, second, and third portions of windings may be formed from a single, continuous instance (e.g., strand, wire, thread, or the like) of conductive medium. A winding may begin by formation of the first portion of windings 166C. After forming the first portion of windings 166C, the conductive medium may be extended at 162C to begin forming the third portion of windings 168. Multiple layers of windings in the third portion are formed as the conductive medium is wound toward the second portion of windings 166D and back toward the first portion of windings 166C any number of times. When the third portion of windings 168 is formed to a desired number of layers, the continuous medium is extended at 162D to form the second portion of windings 166D. Subsequently, a first portion 166A and a second portion 166B of the electromagnetic coil structure 164 are electromechanically affixed (e.g., soldered) to the flexible substrate 102.

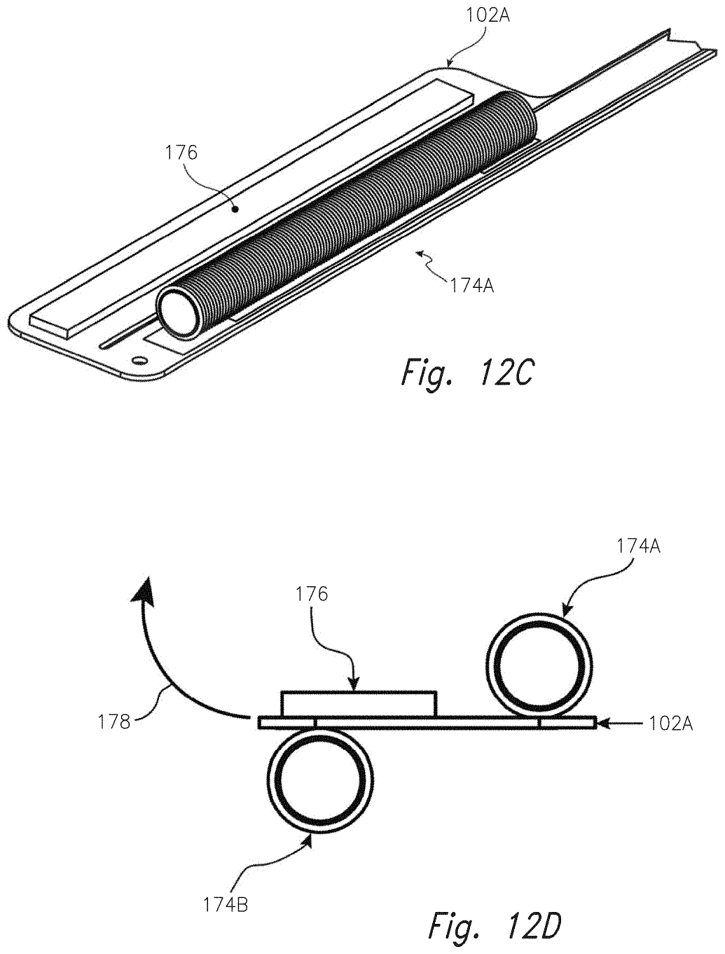

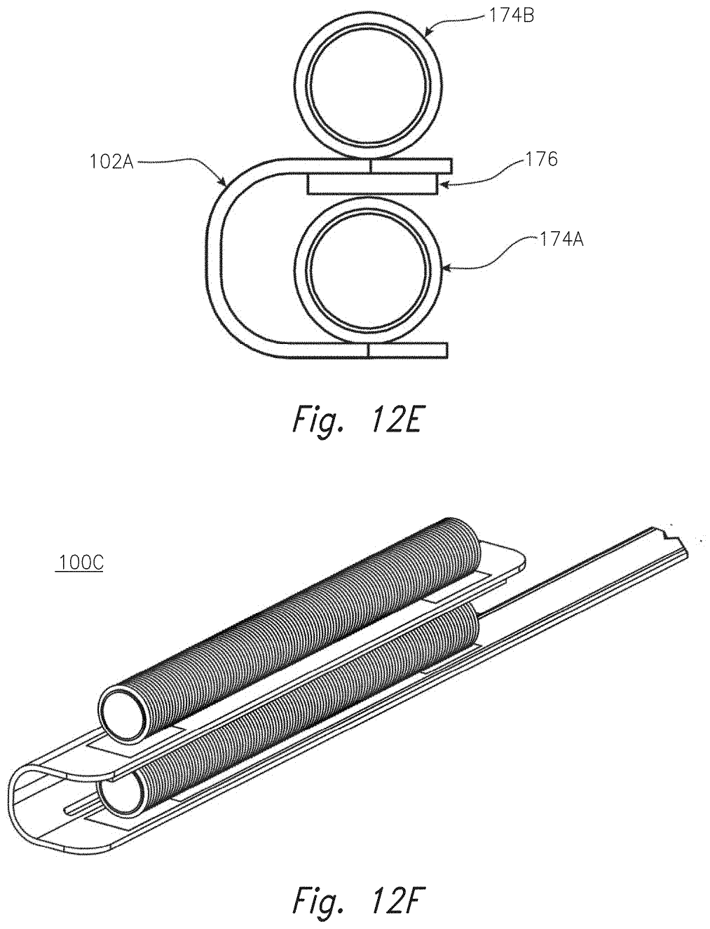

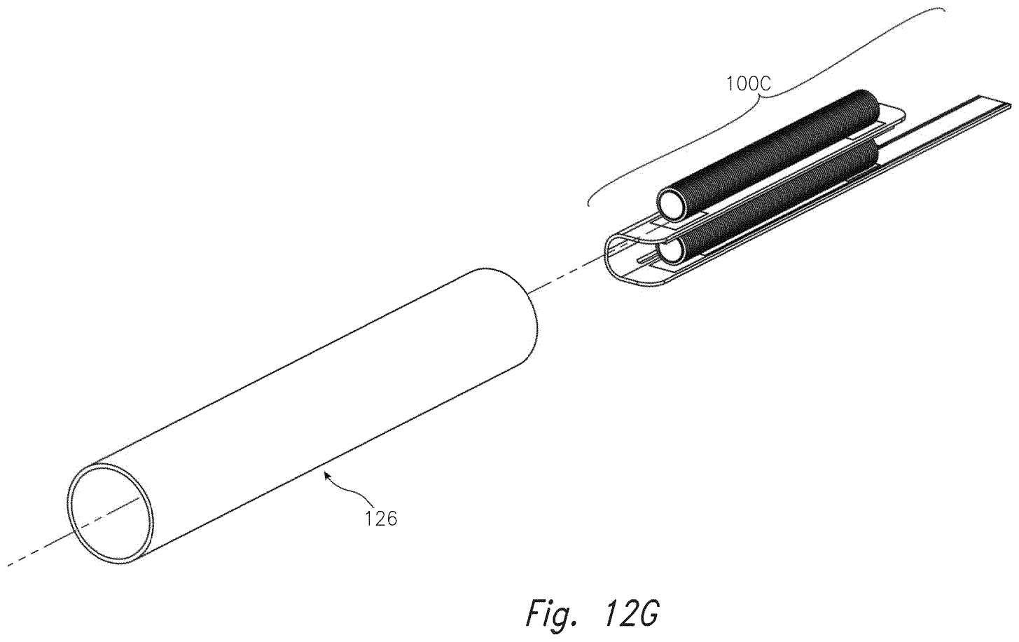

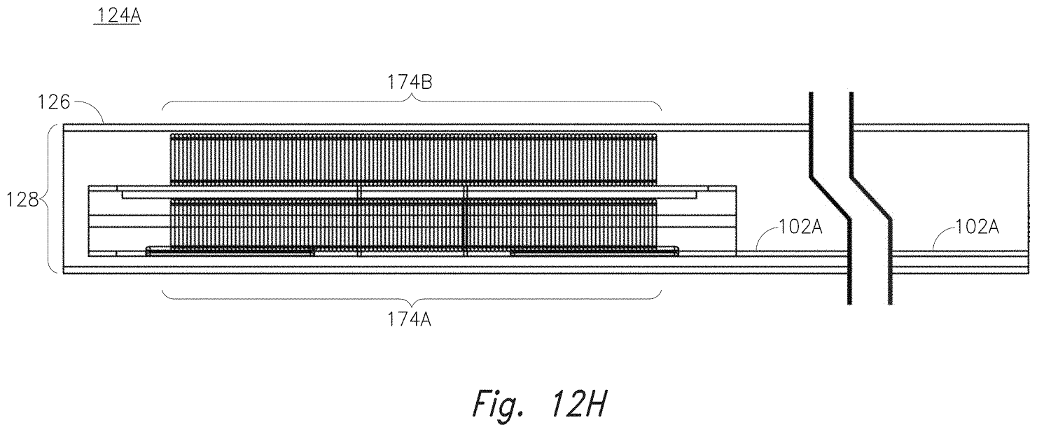

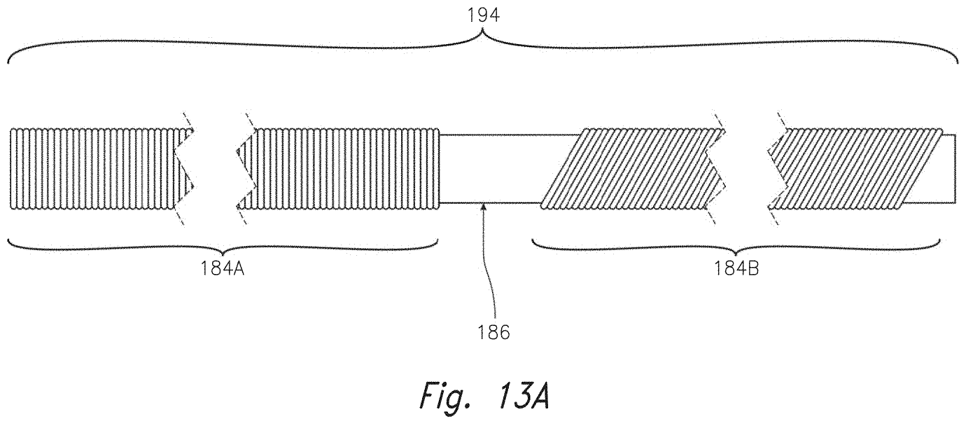

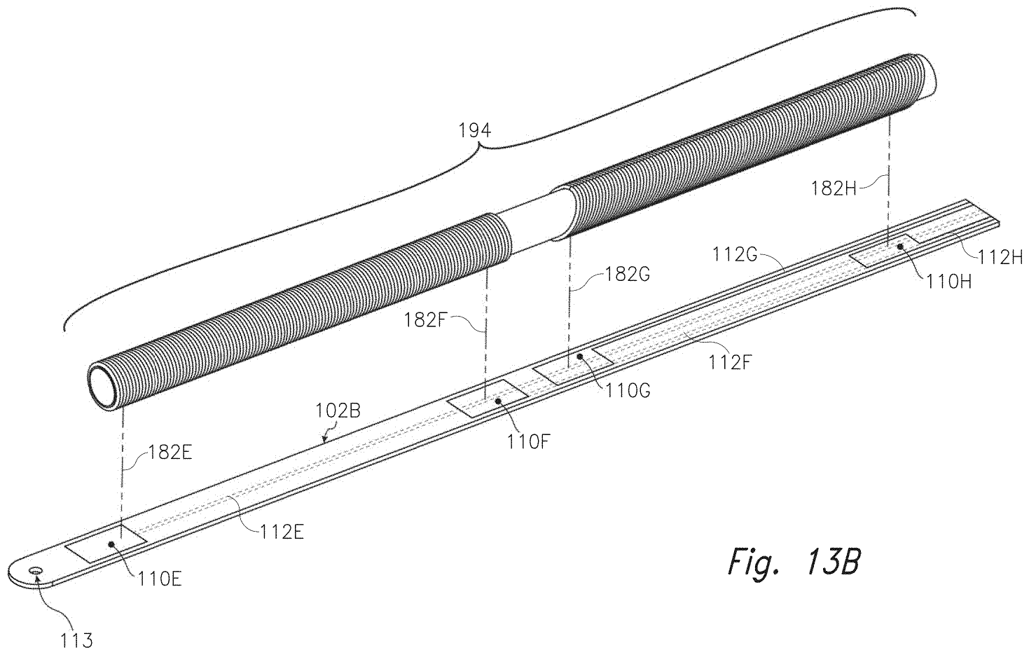

FIGS. 12A-12H illustrate formation and use of another flexible circuit device embodiment 100C. The flexible circuit device embodiment 100C bears a plurality of coils, each having a non-ferrous core. FIGS. 12A-12H may be collectively referred to as FIG. 12.

FIG. 12A is a flexible circuit 102A arranged for acceptance of a plurality of electromagnetic coil structures. Construction, materials, general dimensions, and other characteristics of flexible circuit 102A is along the lines of the flexible circuit 102 presented in this disclosure.

Flexible circuit 102A includes an optional assembly feature, which in FIG. 12 is represented as an aperture 113. Other embodiments of the assembly feature (e.g., holes of different dimensions, protuberances, textures, and the like) are also contemplated. The flexible circuit 102A of FIG. 12 is formed having four conductor regions, including a first and second conductor regions 110A, 110B and third and fourth conductor regions 110C, 110D. Other embodiments may have a different number of conductor regions.

First conductor 110A and second conductor 110B are formed on a first, top surface of the flexible circuit 102A, and third conductor 110C, and fourth conductor 110D, which are represented in dashed lines in FIG. 12A, are formed on a second, bottom surface of the flexible circuit 102A. Each of the conductors is electrically coupled to a conductive trace of flexible circuit 102A. First conductor 110A is electrically coupled to a first conductive trace 112A, and second conductor 110B is electrically coupled to a second conductive trace 112B on the top surface of the flexible circuit 102A. Third conductor 110C is electrically coupled to the third conductive trace 112C, and fourth conductor 110D is electrically coupled to the fourth conductive trace 112D on the opposing bottom surface of the flexible circuit 102A.

In FIG. 12B, a first electromagnetic coil structure 174A is provided along alignment tracks 172A, 172B. A second electromagnetic coil structure 174B is provided along alignment tracks 172C, 172D. Each of the first and second electromagnetic coil structures 174A, 174B may be arranged with the same or similar techniques, materials, dimensions, and other procedures and characteristics as the electromagnetic coil structure 104 of FIG. 2, the electromagnetic coil structure 134 of FIG. 8, and other electromagnetic coil structures described in the present disclosure.

The alignment tracks illustrated in FIG. 12B may be followed manually coupling the electromagnetic coil structures 174A, 174B to the flexible circuit device embodiment 100C (FIG. 12F). Alternatively, the alignment tracks 172A -172B and 172C-172D may be followed in an automated process after forming the respective electromagnetic coil structures 174A, 174B. After the electromagnetic coil structures 174A, 174B are in direct contact with the flexible substrate 102A at their respective conductor regions 110A-110D, the electromagnetic coil structures 174A, 174B are electrically, mechanically, or electromechanically coupled to the flexible substrate 102A. The coupling may be manually performed, performed via a solder re-flow process, performed via a solder bar, or performed in some other way.

In any of several cases, the first electromagnetic coil structure 174A and the second electromagnetic coil structure 174B are joined to the flexible substrate 102A concurrently, sequentially, consecutively, temporally, or in any desirable way. In some cases, a single one of the electromagnetic coils structures 174A, 174B is affixed to the flexible substrate 102A only, and no other electromagnetic coil structures are attached. In other cases, three or more electromagnetic coil structures are affixed to the flexible substrate 102A.

FIG. 12C is a perspective view of the partially formed flexible circuit device embodiment 100C (FIG. 12F). In the figure, a top-side electromagnetic coil structure 174A has been affixed to the flexible substrate 102A. The method of affixation, having been described elsewhere in the present disclosure, is not further described.

Also in FIG. 12C, spacing apparatus 176, which is an optional means of separating a portion of the flexible substrate from an electromagnetic coil structure 174A, is located on the top side of the flexible substrate 102A. The spacing apparatus 176 may be a solid structure, a hollow structure, a segmented structure, a gelatinous structure, a shaped structure, or a structure formed in any desirable way. The spacing apparatus 176 may be formed from plastic, paper, silicon, rubber, an adhesive, or any other selected material having desirable properties (e.g., weight, cost, electrical insulating properties, electrostatic resistance properties, flexibility, adhesion, or the like).

A front-side view of the partially formed flexible circuit device shown in FIG. 12C is illustrated in FIG. 12D. The flexible substrate 102A is identified, the first and second electromagnetic coil structures 174A, 174B are identified, and the optional spacing apparatus 176 is identified. Also identified in FIG. 12D is a direction of travel 178 of one portion of the device when the flexible substrate 102A is folded.

FIG. 12E is another front-side view of the partially formed flexible circuit device wherein the act of folding the flexible substrate 102A into a final position has been completed. The flexible substrate 102A is identified, the first and second electromagnetic coil structures 174A, 174B are identified, and the optional spacing apparatus 176 is identified. In FIG. 12E, the optional spacing apparatus 176 is illustrated as a rigid structure. In other embodiments, the spacing apparatus 176 may include a registration feature (e.g., a shape, an adhesive, an alignment marking, or the like) to desirably mate with or seat the first electromagnetic coil structure 174A.

FIG. 12F is a perspective view of the fully formed flexible circuit device embodiment 100C. The plurality of coils, each having a non-ferrous core, in at least one arrangement of the flexible circuit device embodiment 100C, is shown in FIG. 12F.

FIG. 12G shows a path of travel for a flexible circuit device embodiment 100C as the device will enter a lumen of a catheter 126. The procedure of such use in FIG. 12G, and the path taken by the flexible circuit device embodiment 100C, is along the lines of that described with respect to FIG. 7.

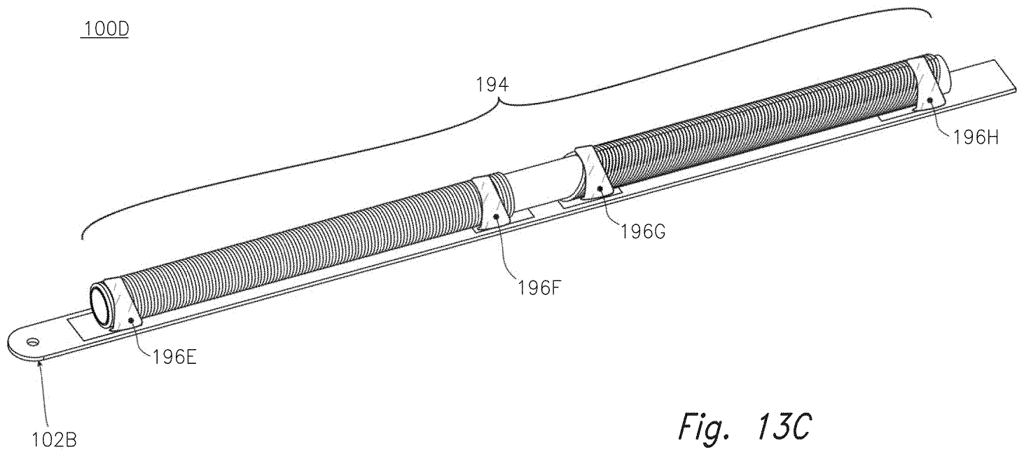

FIG. 12H is a cutaway view 124A embodiment of a flexible circuit device embodiment 100C bearing an two electromagnetic coil structures 174A, 174B within a lumen 128 of a catheter 126 during a medical procedure. FIGS. 13A-13C illustrate formation and use of yet one more flexible circuit device embodiment 100D. The flexible circuit device embodiment 100D bears a plurality of coils sharing a common non-ferrous core 186. FIGS. 13A-13C may be collectively referred to as FIG. 13.

FIG. 13A is a multi-coil electromagnetic coil structure 194 bearing a first electromagnetic coil structure 184A that has a plurality perpendicular coil windings and a second electromagnetic coil structure 184B that has a plurality of canted, off-axis coil windings. The first and second electromagnetic coil structures 184A, 184B share a common non-ferrous core 186. Each of the first and second electromagnetic coil structures 184A, 184B may be arranged with the same or similar techniques, materials, dimensions, and other procedures and characteristics as the electromagnetic coil structure 104 of FIG. 2, the electromagnetic coil structure 134 of FIG. 8, the electromagnetic coil structures 174A, 174B of FIG. 12, and other electromagnetic coil structures described in the present disclosure. Accordingly, it is contemplated that any number of electromagnetic coil structures may be linearly arranged on a common non-ferrous core 186 structure with a same or different number of windings, orientation of windings, number of layers of windings, material forming said windings, and so on.

The non-ferrous core 186 of FIG. 13A may be arranged with the same or similar techniques, materials, dimensions, and other procedures and characteristics as the non-ferrous core structures described in the present disclosure. In addition, or in the alternative, the non-ferrous core 186 of FIG. 13A may have a longer or shorter linear length, a different diameter, a different amount of flexibility, a different rigidity, and the like.

FIG. 13B includes a flexible circuit 102B embodiment arranged for acceptance of a multi-coil electromagnetic coil structure 194. Construction, materials, general dimensions, and other characteristics of flexible circuit 102B is along the lines of other flexible circuits (e.g., flexible circuit 102, flexible circuit 102A) presented in this disclosure. The flexible circuit 102B embodiment includes an optional aperture 113 assembly feature. Other flexible circuit 102B embodiments may or may not include an assembly feature.

The flexible circuit 102B of FIG. 13 is formed having four conductor regions, including a first conductor region 110E, a second conductor region 110F, a third conductor region 110G, and a fourth conductor region 110H. Other flexible circuit embodiments may have a different number of conductor regions. Each of the first, second, third, and fourth conductor regions 110E-110H are formed on a top surface of the flexible circuit 102B, but other flexible circuit arrangements may be formed differently. Each of the conductor regions 110E-110H is electrically coupled to a respective conductive trace 112E-112H of flexible circuit 102B. First conductor region 110E is electrically coupled to a first conductive trace 112E, second conductor region 110F is electrically coupled to a second conductive trace 112F, third conductor region 110G is electrically coupled to the third conductive trace 112G, and fourth conductor region 110H is electrically coupled to the fourth conductive trace 112H. In the embodiment of FIG. 13B, the first and second conductive traces 112E, 112F are formed on the bottom surface of the flexible circuit 102B, and the third and fourth conductive traces 112G, 112H are formed on the top surface of the flexible circuit 102B. In other cases of any of the flexible circuits described in the present disclosure, it is contemplated that conductive traces may be formed on the same or different surfaces of the flexible circuit, within one or more layers of the flexible circuit, or in any other portion of the flexible circuit.

In FIG. 13B, the first electromagnetic coil structure 184A of the multi-coil electromagnetic structure 194 is provided along alignment tracks 182E, 182F, and the second electromagnetic coil structure 184B is provided along alignment tracks 182G, 182H. As described herein, the alignment tracks in FIG. 13B may be followed, and the electromagnetic coils structures may be affixed to the flexible substrate 102B manually, semi-automatically, automatically, or the like.

FIG. 13C is a perspective view of the flexible circuit device embodiment 100D. In the figure, the multi-coil electromagnetic coil structure 194 has been affixed to the flexible substrate 102B. Particularly, a first portion of windings 196E has been electromechanically affixed to the flexible substrate 102B at the first conductor region 110E, a second portion of windings 196F has been electromechanically affixed to the flexible substrate 102B at the second conductor region 110F, a third portion of windings 196G has been electromechanically affixed to the flexible substrate 102B at the third conductor region 110G, and a fourth portion of windings 196H has been electromechanically affixed to the flexible substrate 102B at the fourth conductor region 110H. The electromechanical affixation in FIG. 13B is by solder, but other forms of affixation may also be implemented; such methods of affixation, having been described elsewhere in the present disclosure, are not further described here.

Having now set forth various embodiments, it may further be helpful to an understanding of the invention to set forth some additional definitions of certain terms used herein.

“Medical instrument” refers to a device, instrument, apparatus, constructed element or composition, machine, implement, or similar or related article that can be utilized to diagnose, prevent, treat or manage a disease or other condition(s). For example, medical instruments are used on patients in surgery, preventive care, diagnosis of disease or other condition, treatment, and a wide range of other physiological processes. A medical instrument is a device used in a procedure on the body of a subject (e.g., a patient). Medical instruments include needles, probes, stylets, catheters (e.g., a Peripherally Inserted Central Catheter (PICC)), cannulas, medical tubes, tracheal tubes, rigid tubes, and other such apparatus. Some medical instruments have passages to pass light, fluid, or other therapies. Other medical instruments are solid and pass electricity or mechanical force (e.g., a probe used by a medical practitioner to move or sample a biological mass). Accordingly, in some cases, the medical instrument is a hollow tube-like device. In some cases, the medical instrument is an elongated solid member. In some cases, the medical instrument takes another form.

The medical instrument may be placed through the mouth of the subject or through another of the subject's orifices. Alternatively, the medical instrument may be placed through a surgical incision made by a medical practitioner at some location on the body of the subject. The medical instrument may be placed and moved in other ways. The placement of the medical instrument or a device placed by the medical instrument may be permanent, semi-permanent, or temporary.

The medical instruments provided herein may, depending on the device and the embodiment, be implanted within a subject, utilized to deliver a device to a subject, or utilized externally on a subject. In many embodiments the medical instruments provided herein are sterile and subject to regulatory requirements relating to their sale and use. Representative examples of medical instruments are used in cardio-vascular procedures to implant, for example, cardiovascular devices, implantable cardioverter defibrillators, pacemakers, stents, stent grafts, bypass grafts, catheters and heart valves; they are used in orthopedic procedures to implant, for example, hip and knee prostheses, and spinal implants and hardware (spinal cages, screws, plates, pins, rods and artificial discs); and they are used in a wide variety of procedures that place medical tubes, cosmetic and/or aesthetic implants (e.g., breast implants, fillers). Other representative examples of medical instruments are used to deliver a wide variety of polymers, bone cements, bone fillers, scaffolds, and naturally occurring materials (e.g., heart valves, and grafts from other naturally occurring sources); intrauterine devices; orthopedic hardware (e.g., casts, braces, tensor bandages, external fixation devices, tensors, slings and supports) and internal hardware (e.g., K-wires, pins, screws, plates, and intramedullary devices (e.g., rods and nails)); cochlear implants; dental implants; medical polymers; a wide variety of neurological devices; and artificial intraocular eye lenses. Other uses are also contemplated.

An “electromagnet coil structure” or “electromagnetic coil structure” is a structure that includes one or more devices operable to detect an electromagnetic field. In cases where two or more electromagnet coil structures are formed, some or all of the electromagnet coil structures may be arranged in a determined orientation relative to one or more other electromagnet coil structures. Each electromagnet coil structure is created having a wire-like conductor wound into a coil and a core structure centrally located within the center of the coil. In some cases, two or more electromagnet coil structures may share a core structure. The core structure may be a physical material (e.g., plastic, paper, or the like), or the core structure may be an absence of physical material (e.g., air). For example, a first electromagnet coil structure may be formed by winding a copper-based wire around a ferrous or non-ferrous rod core structure at a first location of the ferrous or non-ferrous rod core structure. In some cases, a second coil of copper-based wire is wound around the ferrous or non-ferrous rod core structure at a second location of the ferrous or non-ferrous rod core structure different from the first location of the ferrous or non-ferrous rod core structure. In some cases, such as cases where the rod core structure is a ferrous rod core structure, the rod core structure is removed prior to deploying the coil or coils that remain.

The “conductive medium” and the “wire-like conductor” of a coil in an electromagnet coil structure may be a wire, a trace manufactured with any type of electronic process (e.g., a semiconductor process, a printed circuit process, and the like), or some other such structure. The conductive medium and the wire-like conductor may have a cross-reference shape that is circumferential, substantially circular, substantially square, octagonal, hexagonal, or having some other cross-section. The conductive medium and the wire-like conductor may be arranged in a coil structure by winding the conductive medium or the wire-like conductor around the core structure. Alternatively, the conductive medium and the wire-like conductor may be arranged in a coil by another process, and the core structure may be later placed centrally in the inner void of the coil. The conductive medium and the wire-like conductor may be formed from copper, a copper alloy, gold, tin, steel, or some other electrically conductive material.

“Contain” in all of its forms refers to one structure being integrated or otherwise located inside another structure. Contain includes encase, enclose, encapsulate, surround, envelop, confine, and other like terms. When a first structure contains a second structure, the containment may be total or partial. For example, a housing may contain an electronic circuit. The housing may have holes, slots, open sides, or other features the allow the some or all of the electronic circuit to be seen without opening or otherwise manipulating the housing. As another example, an insulating jacket may contain a wire, a lumen may contain an electromagnet coil structure, and a conductive coil may contain a non-ferrous-based core structure.

In the present disclosure and the claims appended hereto, a structure described as “substantially cylindrical” includes a cylinder who's volume is defined by Formula 1.

V=πr²h (1)

wherein “V” is volume, “r” is radius, and “h” is height.

In addition, objects in the present disclosure that are substantially cylindrical have a length at least twice as long as the diameter, and the cross section of a substantially cylindrical object may be circular, ovular, octagonal, hexagonal, square, triangular, or even a non-symmetrical shape.

In other contexts, where the terms “substantial” or “about” in any grammatical form are used as modifiers in the present disclosure and any appended claims (e.g., to modify a structure, a dimension, a measurement, or some other characteristic), it is understood that the characteristic may vary by up to 30 percent. For example, where a conductive trace extends substantially along the length of a flexible circuit, the conductive trace is at least 30 percent of the length of the flexible circuit. As another example, an electromagnetic coil structure that is about two inches long includes a structure that is exactly two inches long. Different from the exact precision of the term, “two inches,” the use of “about” to modify the characteristic permits a variance of the “two inches” characteristic by up to 30 percent. Accordingly, an electromagnetic coil structure that is “about two inches” long includes devices that are between 1.6 inches long and 2.4 inches long. An electromagnetic coil structure that is 1.5 inches long or less, and an electromagnetic coil structure that is 2.5 inches long or more, however, is not “about two inches” long.

In the present disclosure, conjunctive lists make use of a comma, which may be known as an Oxford comma, a Harvard comma, a serial comma, or another like term. Such lists are intended to connect words, clauses or sentences such that the thing following the comma is also included in the list.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either or both of those included limits are also included in the invention.

Unless defined otherwise, the technical and scientific terms used herein have the same meaning as commonly understood by one of ordinary skill in the art to which this invention belongs. Although any methods and materials similar or equivalent to those described herein can also be used in the practice or testing of the present invention, a limited number of the exemplary methods and materials are described herein.

In the foregoing description, certain specific details are set forth in order to provide a thorough understanding of various disclosed embodiments. However, one skilled in the relevant art will recognize that embodiments may be practiced without one or more of these specific details, or with other methods, components, materials, etc. In other instances, well-known structures associated with electronic and computing systems including client and server computing systems, as well as networks, have not been shown or described in detail to avoid unnecessarily obscuring descriptions of the embodiments.

Unless the context requires otherwise, throughout the specification and claims which follow, the word “comprise” and variations thereof, such as “comprises” and “comprising,” are to be construed in an open, inclusive sense, e.g., “including, but not limited to.”

Reference throughout this specification to “one embodiment” or “an embodiment” and variations thereof means that a particular feature, structure, or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

As used in this specification and the appended claims, the singular forms “a,” “an,” and “the” include plural referents unless the content clearly dictates otherwise. It should also be noted that the term “or” is generally employed in its broadest sense including “and/or” unless the content clearly dictates otherwise.

The headings and Abstract of the Disclosure provided herein are for convenience only and do not interpret the scope or meaning of the embodiments.

The various embodiments described above can be combined to provide further embodiments. These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. An electromagnetic coil device, comprising:
a non-ferrous core having a substantially cylindrical shape;
an insulated conductive medium arranged as a plurality of windings coiled around the non-ferrous core, wherein the insulated conductive medium includes a conductive medium encased within an insulating medium, wherein the plurality of windings include a first portion of windings and a second portion of windings separated by a third portion of windings, and wherein the insulating medium has at least one breakdown characteristic that permits shorting between individual conductors of the conductive medium in the first and second portions of windings thereby creating a first conductor region and a second conductor region electrically separated by the third portion of windings.

2. An electromagnetic coil device according to claim 1, comprising:
a flexible printed circuit having a length and a width, wherein the length is at least twenty times the width, the flexible printed circuit including:
a first conductive trace running substantially along the length of the flexible printed circuit, the first conductive trace having a first end electrically coupled to the first conductor region; and
a second conductive trace running substantially along the length of the flexible printed circuit, the second conductive trace having a first end electrically coupled to the second conductor region.

3. An electromagnetic coil device according to claim 2, wherein the breakdown characteristic is a melting point that is below a temperature of liquid solder, and wherein the electrical coupling of the first and second portions to the first and second conductor regions, respectively, is via a solder connection.

4. An electromagnetic coil device according to claim 1, wherein the breakdown characteristic is a chemical composition that permits separation of the insulating medium from the conductive medium via a chemical reaction, or wherein the breakdown characteristic is a tensile composition that permits separation of the insulating medium from the conductive medium via ultrasound.

5. An electromagnetic coil device according to claim 1, wherein the third portion of windings is a multi-layer portion of windings.

6. An electromagnetic coil device according to claim 1, wherein the third portion of windings has a pitch of about 30 to 60 degrees off of an axis of the non-ferrous core.

7. An electromagnetic coil device according to claim 1, wherein the first, second, and third portions of windings form a continuous set of windings.

8. An electromagnetic coil device according to claim 1, wherein the first portion of windings are electrically coupled to a first end of the third portion of windings via a first conductive conduit, and wherein the second portion of windings are electrically coupled to a second end of the third portion of windings via a second conductive conduit.

9. An electromagnetic coil device according to claim 1, wherein the third portion of windings has a linear length of between about 0.006 inches and 0.125 inches.

10. An electromagnetic coil device according to claim 1, wherein the third portion of windings has an outside diameter of between about 0.0025 inches and two (2) inches in linear length.

11. An electromagnetic coil device according to claim 1, wherein the non-ferrous core is a hollow core.

12. An electromagnetic coil device according to claim 1, wherein the non-ferrous core is a ceramic core, a resin core, or a glass core.

13. A method of operating a medical device, comprising:
passing a distal end of the medical device into a body of a patient while a proximal end of the medical device remains outside the body of the patient, the distal end of the medical device including:
a non-ferrous core having a substantially cylindrical shape; and
an insulated conductive medium arranged as a plurality of windings coiled around the non-ferrous core, wherein the insulated conductive medium includes a conductive medium encased within an insulating medium, wherein the plurality of windings include a first portion of windings and a second portion of windings separated by a third portion of windings, and wherein the insulating medium has at least one breakdown characteristic that permits shorting between individual conductors of the conductive medium in the first and second portions of windings thereby creating a first conductor region and a second conductor region electrically separated by the third portion of windings;
generating an excitation signal from a current induced in the third portion of windings arranged at the distal end of the medical device by an electromagnetic field; and
operating ancillary circuitry arranged at the proximal end of the medical device to detect the excitation signal generated in the third portion of windings, the excitation signal passed via first and second conductive traces running substantially along a length of a flexible printed circuit, wherein the first conductor region is electrically coupled to a first end of the first conductive trace and the second conductor region is electrically coupled to a first end of the second conductive trace.

14. A method according to claim 13, further comprising;
based at least in part on the detected excitation signal, generating a representation of the distal end of the medical device in the body of the patient; and
communicating the representation of the distal end of the medical device in the body of the patient to a presentation system.

15. A method according to claim 14, further comprising;
advancing the distal end of the medical device further into the body of the patient; and
tracking the distal end of the medical device as it advances into the body of the patient.

16. A method according to claim 13, wherein passing the distal end of the medical device into the body of the patient includes passing the passing the distal end of the medical device through a lumen of a catheter.

17. A method of manufacturing a medical device, comprising:
providing a non-ferrous core having a substantially cylindrical shape;
winding an insulated conductive medium around the non-ferrous core a plurality of times, wherein the insulated conductive medium includes a conductive medium encased within an insulating medium, wherein the winding creates a first portion of windings and a second portion of windings separated by a third portion of windings;
creating a first breakdown condition to exceed a breakdown characteristic of the insulating medium that shorts together individual conductors of the conductive medium in the first portion of windings thereby creating a first conductor region; and
creating a second breakdown condition to exceed the breakdown characteristic of the insulating medium that shorts together individual conductors of the conductive medium in the second portion of windings thereby creating a second conductor region.

18. A method according to claim 17, comprising:
providing a flexible printed circuit having a length and a width, wherein the length is at least twenty times the width, the flexible printed circuit including first and second conductive traces running substantially along the length of the flexible printed circuit;
electrically coupling a first end of the first conductive trace to the first conductor region; and
electrically coupling a first end of the second conductive trace to the second conductor region.

19. A method according to claim 18, wherein creating the first breakdown condition includes soldering the first end of the first conductive trace to the first conductor region and creating the second breakdown condition includes soldering the first end of the second conductive trace to the second conductor region.

20. A method according to claim 17, wherein winding the insulated conductive medium around the non-ferrous core the plurality of times includes winding the insulated conductive medium in a plurality of layers.
